(19) United States
(12) Patent Application Publication
(10) Pub. No.: US 2005/0059153 A1
(43) Pub. Date: Mar. 17, 2005

(54) Electromagnetic activation of gene expression and cell growth

(76) Inventors: Frank R. George, Scottsdale, AZ (US); John Moffett, Phoenix, AZ (US)

Correspondence Address: Cathryn Campbell;McDERMOTT, WILL & EMERY, Suite 700, 4370 La Jolla Village Drive, San Diego, CA, 92122, US

(21) Appl. No.: 10/759,526

(22) Filed: Jan. 16, 2004

Related U.S. Application Data
(60) Provisional application No. 60/509,061, filed on Jan. 22, 2003.

(51) Int. Cl. A61N 1/00; C12N 15/01

(52) U.S. Cl. 435/446; 607/88

(57) ABSTRACT

The invention is directed to a method for accelerating the cell cycle by delivering to a cell an effective amount of electromagnetic energy. The invention also provides a method for activating a cell cycle regulator by delivering to a cell an effective amount of electromagnetic energy. Also provided by the invention is a method for activating a signal transduction protein; a method for activating a transcription factor; a method for activating a DNA synthesis protein; and a method for activating a Receptor. A method for inhibiting an angiotensin receptor as well as a method for reducing inflammation also are provided by the present invention. The invention also is directed to a method for replacing damaged neuronal tissue as well as a method for stimulating growth of administered cells.

This application is based on, and claims the benefit of, U.S. Provisional Application No. 60/______ (yet to be assigned), filed Jan. 22, 2003, which was converted from U.S. Ser. No. 10/350,313, and entitled ELECTROMAGNETIC ACTIVATION OF GENE EXPRESSION AND CELL GROWTH, and which is incorporated herein by reference.

### BACKGROUND OF THE INVENTION

This invention relates generally to methods for modulating the activity of gene products in a cell and, more specifically, to methods for modulating the activity of gene products that regulate tissue repair and cell proliferation by delivering electromagnetic energy to cells.

The normal development of all multicellular organisms relies on the orchestrated regulation of when and where each cell proliferates. For example, the formation of the intricate anatomical features of internal organs or the proper migration of nerves throughout the body require that each participating cell sense its environment and respond appropriately to developmental cues. The requirement for regulated proliferation is equally important for the proper functioning of the mature multicellular organism. The average adult human eradicates 50-70 billion cells in the body each day, and a commensurate number of replacement cells must be produced daily. The number and type of cells that are induced to proliferate as replacements depends upon the circumstances under which the original cells were eradicated and the tissues affected.

Harnessing the body's ability to regulate spatial and temporal aspects of cell proliferation is one approach to treating diseases and conditions characterized by traumatic or pathogenic tissue destruction. Growth factors have been considered candidate therapeutics for treating a number of such conditions because they are synthesized by and stimulate cells required for tissue repair, and are deficient in a number of chronic conditions. With the understanding that defects in growth factor signaling contribute to the development and/or persistence of a number of chronic conditions, it is logical to conclude that reinstitution or normalization of that signaling would promote healing. Although there is some evidence that pharmacological application of growth factors enhances healing in some conditions such as wound repair, it is often difficult to achieve targeted delivery of growth factors in such a way that healthy tissues are not inadvertently stimulated.

In particular, clinical studies of growth factor use in wound repair have been disappointing. The lack of therapeutic efficacy may be in part due to the complexity of the programmed sequence of cellular and molecular events involved in wound healing, including macrophage activation during inflammation, cell migration, angiogenesis, provisional matrix synthesis, synthesis of collagen by fibroblasts, and re-epithelialization. Similarly complex sequences of cellular events are invoked during the repair of damage to tissues in response to other diseases and conditions. Current pharmaceutical approaches do not fully mimic the necessary spatial and temporal patterns of cellular regulation and activity needed to promote cell proliferation for healing in most biological contexts.

The ability to control cell proliferation is also important for growth of cells in culture for applications such as bioindustrial processing. Cultures of genetically engineered animal cells are currently used to produce post-translationally modified and physiologically active proteins for use as pharmaceutical agents. Cell culture for pharmaceutical protein production in many cases is an expensive, slow process due to the complex media required and the slow rate of cell proliferation. Animal cells usually require mitogenic stimulation to proliferate. This mitogenic stimulation is often provided by growth factors, which are supplied to the medium either as purified proteins or by the addition of animal blood sera.

The use of animal blood sera as a mitogen causes a number of problems but nevertheless is used currently in biotechnological manufacturing processes employing animal cells. There is a risk that fetal blood sera will contain unwanted biological agents such as viruses, mycoplasma and prions, which if not properly removed or avoided can contaminate the final pharmaceutical preparation and infect a patient. The screening of animal blood sera for viruses and mycoplasma is feasible but expensive and complicated. Furthermore, inactivation of these contaminants by heating the serum often comes at the cost of inactivating valuable growth factors.

The use of purified growth factor proteins as mitogens in cell culture, although providing advantages over the use of animal blood sera, is out of reach for many systems. The number and type of growth factors that stimulate a particular animal cell to grow are not known in many cases. Even in cases where a useful growth factor has been identified, purified preparations are often required in large quantities. In this regard, 10,000 liter reactors are not unusual for the culture of mammalian cells producing therapeutic proteins. The time and resources required to produce sufficient amounts of growth factors to sustain reactor cultures at these levels can be prohibitive.

Thus, there exists a need for methods of stimulating cell proliferation and associated cellular processes in vivo and in vitro. The present invention satisfies this need and provides related advantages as well.

### SUMMARY OF THE INVENTION

The invention is directed to a method for accelerating the cell cycle by delivering to a cell an effective amount of electromagnetic energy. The invention also provides a method for activating a cell cycle regulator by delivering to a cell an effective amount of electromagnetic energy. Also provided by the invention is a method for activating a signal transduction protein; a method for activating a transcription factor; a method for activating a DNA synthesis protein; and a method for activating a Receptor. A method for inhibiting an angiotensin receptor as well as a method for reducing inflammation also are provided by the present invention. The invention also is directed to a method for replacing damaged neuronal tissue as well as a method for stimulating growth of administered cells.

### BRIEF DESCRIPTION OF THE DRAWINGS

FIG. 1 shows the growth stimulation of untreated human dermal fibroblasts (HDF) by media transferred from HDF cells exposed to electromagnetic energy. FIG. 1A shows the growth response of the HDF cells from which the medium was transferred from at the hours shown. FIG. 1B shows the induction of proliferation of untreated cells to which the media from the cells in FIG. 1A was transferred.

FIG. 2 shows a Western blot that demonstrates significant activation of ERK-1 (p44) and ERK-2 (p42) after the initiation of treatment with electromagnetic energy.

FIG. 3 shows incorporation of BrdU as an indicator of entry into S phase of HDF cells stimulated with electromagnetic energy.

FIG. 4 shows two autographs that show gene expression in human diploid fibroblasts. Each array contains 1,176 known cDNA sequences involved in tissue repair, cell cycle and cell growth. The Black arrows are examples of genes that are not increased in expression following treatment with electromagnetic energy. The Grey arrows are examples of genes significantly up-regulated in treated cells. The brackets at the bottom of the arrays indicate control cDNA sequences used to normalize samples.

FIG. 5 shows autographs of the analysis of cDNA sequences implicated in inflammation processes. Each array contains 234 cDNAs in duplicate. The Black arrows are examples of genes that are not increased in expression following electromagnetic treatment. The Grey arrows are examples of genes significantly up-regulated in Provant treated cells. The brackets at the bottom of the arrays indicate control cDNA sequences used to normalize samples.

FIG. 6 shows expression profiles of genes in HDF cells treated with electromagnetic energy. In FIG. 6A the genes representing the entire set of fibroblast microarray data are grouped into clusters representing similarity of expression patterns, regardless of function. The functional groupings in panels 6B-6D represent genes selected from groups of genes whose function is important to cell division and/or wound healing. Both sets of data are arranged so that early expression genes are displayed first, followed by intermediate expression and late expression. The scale in each panel (0 to 8) represents the ratio of the raw expression level for the experimental time point to the expression level in a non-treated control scenario. For example, dark shading means an eight-fold induction over control. FIG. 6B shows expression levels of genes divided into the following functional groups: Adhesion Molecules; Cyclins; DNA Synthesis Proteins; and Growth Factors and corresponding Receptors. FIG. 6C shows expression levels of genes divided into the following functional groups: Interleukins, Interferons and corresponding Receptors; MAP Kinases; other kinases; and Matrix Metalloproteinases and their Inhibitors. FIG. 6D shows expression levels of genes divided into the following functional groups: Protein Kinase Cs; Tumor Necrosis Factors and their Receptors; and Transcription Factors. Measurements for all genes were for expression between 5 minutes and eight hours post-treatment.

FIG. 7 shows expression profiles of genes in human keratinocytes treated with electromagnetic energy. In FIG. 7A the genes representing the entire set of keratinocyte microarray data are grouped into clusters representing similarity of expression patterns, regardless of function. The functional groupings in panels 7B-7D represent genes selected from groups of genes whose function is important to cell division and/or wound healing. Both sets of data are arranged so that early expression genes are displayed first, followed by intermediate expression and late expression. The scale in each panel (0 to 8) represents the ratio of the raw expression level for the experimental time point to the expression level in a non-treated control scenario. For example, dark shading means an eight-fold induction over control. FIG. 7B shows expression levels of genes divided into the following functional groups: Adhesion Molecules; Cyclins; DNA Synthesis Proteins; and Growth Factors and corresponding Receptors. FIG. 7C shows expression levels of genes divided into the following functional groups: Interleukins, Interferons and corresponding Receptors; MAP Kinases; other kinases; and Matrix Metalloproteinases and their Inhibitors. FIG. 7D shows expression levels of genes divided into the following functional groups: Protein Kinase Cs; Tumor Necrosis Factors and their Receptors; and Transcription Factors. Measurements for all genes were for expression between 5 minutes and eight hours post-treatment.

### DETAILED DESCRIPTION OF THE INVENTION

The present invention is based on the discovery that stimulation of cells with electromagnetic energy modulates the activity of genes involved in tissue repair and cell growth and the cellular levels of gene products that are involved in molecular regulatory networks. As demonstrated herein, stimulation with electromagnetic energy modulates the levels of gene products such as extracellular matrix receptors, signal transduction proteins, cell cycle regulators, transcription factors and nucleic acid synthesis proteins. The changes to these regulatory networks lead to changes in cellular functions that include, but are not limited to, acceleration of the cell cycle, stimulation of wound healing, stimulation of cell proliferation, stimulation of tissue growth, and modulation of inflammatory responses. Accordingly, the invention provides methods for delivering to a cell an effective amount of electromagnetic energy to change such cellular functions. Furthermore, the invention provides diagnostic methods for monitoring the cell cycle, wound healing, tissue growth, or inflammation by determining a level of a gene product involved in a regulatory network.

The invention is further based on the discovery that delivery of electromagnetic energy to a resting cell accelerates the cell cycle, not only by inducing entry into the cell cycle, but also by reducing duration of the cell cycle. In particular, the gap, G₁ phase, that intervenes between the formation of a daughter cell by mitosis, M phase, and DNA synthesis, S phase, is shortened by delivering electromagnetic energy in accordance with a method of the invention. Accordingly, the invention provides a method for accelerating the cell cycle of a population of cells by delivering electromagnetic energy to the population of cells. In particular embodiments, a method for stimulating proliferation of a population of cells can be used in vitro, for example, to produce replacement tissues, or in vivo, for example, to stimulate introduction of therapeutic cells or to stimulate replacement of damaged cells such as at the site of a wound.

As used herein, the term “cell cycle” is intended to mean the process of cell replication occurring between the formation of a cell by division from its mother cell and its division to form two daughter cells. The cell cycle can be divided into a number of periods typically identified as M phase, which is the period of mitosis and cell division; G₁, which is the gap period occurring after telophase of mitosis and prior to S phase; S phase, which is the period of DNA synthesis occurring after G₁ and before G₂; and G₂, which is the gap period after S phase and before prophase of mitosis.

As used herein, the term “accelerate,” when used in reference to the cell cycle, is intended to mean decreasing the period of time for the cell cycle in a replicating cell. A replicating cell is a cell that is in M, G₁, S or G₂ phase. In contrast, a non-replicating cell is a cell that is in the resting phase known as G₀ phase. A decrease in the period of time can include a decrease in the period of time spent in the G₁, G₂ or S phase.

As used herein, the term “electromagnetic energy” is intended to mean a form of energy having both electric and magnetic components and properties of wavelength and frequency. Forms of energy included in the term are, for example, X-ray radiation, which has a wavelength in the range of about 0.05 to 100 angstroms; ultraviolet radiation, which has a wavelength in the range of about 200 to 390 nm; visible radiation, which has a wavelength in the range of about 391 to 770 nm; infrared radiation, which has a wavelength in the range of about 0.771 to 25 microns; microwave radiation, which has a wavelength in the range of about 1 millimeter to 1 meter; and radiofrequency radiation, which has a wavelength greater than about 1 meter.

As used herein, the term “cell cycle regulator” is intended to mean a molecule that activates or inhibits progression through the cell cycle. A molecule included in the term can activate progression through the cell cycle by initiating the cell cycle or a phase of the cell cycle or by increasing the rate of the cell cycle or a phase of the cell cycle. A molecule included in the term can inhibit progression through the cell cycle by stopping the cell cycle or a phase of the cell cycle or by decreasing the rate of the cell cycle or a phase of the cell cycle. Examples of molecules included in the term are cyclins such as Cyclin H; cyclin dependent kinases such as CDKN2D, CDK7, CDK5 and CDK6; CLK1; CKS2; LHX1; Cyclin 6 Kinase; Cell Cycle Regulated Kinase; CDK inhibitors and CDC20.

As used herein, the term “signal transduction protein” is intended to mean a protein that converts input energy of one form to output energy of another form in a regulatory network of a cell. The term can include, for example, a kinase, phosphatase, or G-protein. Other examples of proteins included in the term are MAP3K11, MAPK7/ERK5, MAPK5/MEK5, MEK1, MEK2, MEK3, MAP kinase p38, BDIIF Tyr Kinase, Serine Kinase, p68 Kinase, PAK2 and SPS1/ste20

As used herein, the term “transcription factor” is intended to mean a protein that initiates or regulates synthesis of RNA when in the presence of a DNA template and RNA polymerase. Examples of proteins included in the term include TFIIB 90-Kd, C-jun, Est1, and Early Response Protein.

As used herein, the term “DNA synthesis protein” is intended to mean a protein that catalyzes or facilitates formation of a bond between nucleotides of a deoxyribonucleic acid polymer. Examples of proteins included in the term are helicases such as DNA Helicase A, ligases such as DNA Ligase 1, DNA Polymerases such as DNA Polymerase Delta, topoisomerases such as Topoisomerase I, and DNA Repair Enzymes.

As used herein, the term “receptor” is intended to mean a protein that binds to a molecule and transduces a signal that alters cell function. A protein included in the term can be a soluble protein or membrane protein. Examples of proteins included in the term are the Angiotensin Receptor, Tyrosine Kinase Receptor, Thrombin Receptor, Adenosine A1 Receptor, Na/H Exch, Ephrin A Receptor, Insulin Receptor, Cell-Cell Adhesion Protein, Matrix Adhesion Protein, ICAM1, H₂O Channel, Integrinβ8, K⁺ Channel, Glucose Transporter, TGFβ Receptor, PDGF Receptor, Cl⁻ Channel, TNF Receptor, IGFBP1, Ras Homolog, RAS Associated Protein, RAS GTPase, RAB6, RAB5A, Ca⁺² Adenylylcyclase, Adenylylcyclase, Protein Kinase C and S100 Ca⁺² Binding Protein.

As used herein, the term “activating,” when used in reference to a gene product, is intended to mean increasing the activity of the gene product. The activity can be increased, for example, by increasing the expression of the gene product, decreasing degradation of the gene product, increasing the catalytic rate of the gene product or increasing affinity of the gene product for its substrate.

As used herein, the term “tissue” is intended to mean a group of cells united to perform a particular function. A group of cells included in the term can further form an ordered structure such as a tube or sheet. Alternatively a group of cells can be unstructured, for example, occurring in mass or clump. Examples of tissues include epithelial, connective, skeletal, muscular, glandular, and nervous tissues.

As used herein, the term “stimulating growth” is intended to mean initiating or increasing the rate at which cells proliferate. The term can include, for example, accelerating the cell cycle, initiating entry into the cell cycle, or leaving G₀ or the resting state.

As used herein, the term “wound” is intended to mean a stress to a tissue due to injury. A stress to a tissue can involve a breach and included in the term can be a chronic wound, pressure ulcer, diabetic ulcer, venous stasis ulcer, burn or trauma. The term can include a breach that is at a particular stage of healing including, for example, an inflammatory phase in which leukocytes migrate to the wound site and monocytes are converted to macrophages; proliferative phase in which granulation occurs due to proliferation of fibroblasts, production of a collagen matrix and vascularization; epithelialization phase in which epithelial cells grow along fibrin and myofibroblasts synthesize collagen; or differentiation phase in which collagen is degraded and resynthesized as the tissue is remodeled.

As used herein, the term “matrix” is intended to mean a substrate capable of supporting a population of proliferating cells. The term can include, for example, a synthetic substrate or polymer such as nylon (polyamides), dacron (polyesters), polystyrene, polypropylene, polyacrylates, polyvinyl compounds (e.g., polyvinylchloride), polycarbonate (PVC), polytetrafluorethylene (PTFE; teflon), thermanox (TPX), nitrocellulose or polyglycolic acid (PGA). Also included in the term is a biological matrix such as cotton, cat gut sutures, cellulose, gelatin, dextran or an in vivo site such as a tissue or wound.

As used herein, the term “level,” when used in reference to a molecule, is intended to mean an amount, concentration, or activity of the molecule. An amount or concentration included in the term can be an absolute value such as a molar concentration or weight or a relative value such as a percent or ratio compared to one or more other molecules in a sample. An activity can be an absolute value such as a turnover number, reaction rate, or binding constant or a relative value such as a percent or ratio compared to one or more other molecules.

The invention provides a method for accelerating the cell cycle. The method includes a step of delivering to a cell an effective amount of electromagnetic energy to accelerate the cell cycle of the cell.

The methods of the invention provide for acceleration of the cell cycle such that cells that are actively replicating do so at a faster rate. The cell cycle is accelerated in the methods at least in part by a reduction in the duration of the G₁ stage of the cell cycle. When the cell cycle is accelerated for a replicating cell, the rate at which the cell completes the cell cycle and replicates its DNA is increased. Generally, a population of cells can include cells that are replicating in the cell cycle, resting in G₀, or a combination of cells in both states. For a population that includes resting cells in the G₀ state, growth of the population can be stimulated by inducing the resting cells to enter the cell cycle and become replicating cells. A mixture of cells containing both resting and cycling cells can be stimulated and growth increased due to both acceleration of the cell cycle for cells that are replicating as well as recruitment of resting cells into the cell cycle. However, acceleration of the cell cycle provides a different means of increasing the rate at which a population of cells grows compared to recruitment of cells into the cell cycle. As set forth in further detail below, acceleration of and recruitment into the cell cycle can be induced in a method of the invention by modulating the activity of molecular regulatory networks controlling the cell cycle.

Acceleration of the cell cycle will result in a decrease in the period of time for the cell cycle of a treated cell compared to an untreated cell. An effective amount of electromagnetic energy can be delivered in accordance with the methods described herein to accelerate the cell cycle to achieve a desired rate of cell proliferation. In some applications of the methods an effective amount of electromagnetic energy can be delivered to cause a 10%, 25%, 50% or 75% increase in the cell cycle. When a faster rate of cell proliferation is desired, an effective amount of electromagnetic energy can be delivered resulting in, for example, a 2 fold, 3 fold, 4 fold, 5 fold or higher increase in the cell cycle. An untreated cell used for comparing a cell that has been contacted with electromagnetic energy can be any cell that is not influenced by treatment with electromagnetic energy including, for example, the cell itself prior to delivery of electromagnetic energy or a control cell that is not treated with an effective amount of electromagnetic energy to accelerate the cell cycle. The magnitude of cell cycle acceleration can be influenced by altering parameters of electromagnetic energy delivered in a method of the invention as set forth in further detail below.

Electromagnetic energy is delivered to a cell using any apparatus capable of generating and applying known dosages of electromagnetic energy of defined specifications to the cell. Generally, an apparatus useful in the invention for delivering electromagnetic energy to a cell will include an electromagnetic energy generator, a treatment applicator that delivers energy from the generator to a cell and a device for controlling the amount or characteristics of the electromagnetic energy delivered by the applicator. An exemplary electromagnetic energy treatment apparatus that can be used in a method of the invention is described in U.S. Pat. No. 6,344,069 B1, which describes an apparatus that includes a pulsed electromagnetic energy generator; a power controller, including a power level controller responsive to signals from multiple sensing and control circuits; and a treatment pad applicator.

The parameters under which electromagnetic energy is delivered to a cell can be adjusted to suit a particular application of the methods. Exemplary parameters that can be adjusted include, without limitation, wavelength, power level, duration of delivery, delivery of constant output or pulsed output and, if pulsed output is used, pulse rate and pulse width. A power level in the range of about 1 to 300 mw/cm² (60 to 1,065 V/m) is useful in a method of the invention. The pulse rate can be any in the range of about 100-3,600 ppm (pulses per minute), while pulse width is typically in the range of about 5-300 microseconds. The wavelength or frequency of the electromagnetic energy can be in a range selected from X-ray radiation, ultraviolet radiation, visible radiation, infrared radiation, microwave radiation, radiofrequency radiation, or combination thereof. Typically, the electromagnetic energy is delivered under parameters in which the cell being treated does not sustain substantial DNA damage.

As an exemplary application, parameters that are effective for acceleration of the cell cycle for treatment of wounds include delivery of RF frequency energy with an average power of about 15 mw/cm², 32 mw/cm², or 100 mw/cm² (about 240 V/m, 350 V/m or 600 V/m) pulse envelopes with a duration of about 32 microseconds and a repetition rate of about 1,000 pulses per second. For example, in treating pressure ulcers, power of the RF energy can about 30-40 mw/cm² (335-390 V/m) with a pulse envelope having a duration between about 16-20 microseconds and a repetition rate between about 1,200-1,500 pulses per second. In another effective embodiment, RF energy is delivered with a repetition rate in the range of about 900-1,200 pulses per second and a duration of about 30-45 microseconds, giving an output of in the range of about 30-65 mw/cm² (335-500 V/m) average power. In yet another embodiment, RF energy is delivered with a repetition rate in the range of about 600-1,000 pulses per second and a pulse duration in the range of about 32-60 microseconds, giving an output in the range of about 30-100 mw/cm² (335-600 V/m) average power. Other parameters useful in the invention are demonstrated in the Examples provided below. The parameters exemplified above with respect to wound healing can be used in other applications of the methods such as reducing inflammation, stimulating cell proliferation, accelerating the cell cycle, modulating the activity of a gene product or replacing a damaged tissue.

Another parameter that can be adjusted is the number of electromagnetic energy deliveries given to a cell during a specified time period. Electromagnetic energy can be delivered in a single administration or in multiple. Multiple deliveries can be administered over a time period of minutes, hours, days or weeks. For example, an effective treatment profile for wound healing is described in U.S. Pat. No. 6,344,069 B1 and includes delivery of electromagnetic energy twice a day, eight to twelve hours between treatments, for thirty minutes per treatment.

The parameters for delivery of electromagnetic energy for a particular application of the methods can be determined based on a dose-response analysis. Those skilled in the art will know or be able to determine an appropriate response that indicates a favorable outcome for a particular application such as treatment of a disease or condition and will be able to systematically vary the parameters while evaluating the response as it correlates with a desired outcome. Exemplary diseases and conditions that can be treated using a method of the invention and responses that are indicative of a favorable outcome are set forth in further detail below. A further response that can be monitored in a dose-response analysis is expression of particular genes or activity of gene products, which is also set forth in further detail below.

The invention provides a method for delivering an effective amount of electromagnetic energy to modulate the activity of a cellular component. The activity of a cellular component can be modulated by increasing or decreasing the level of the cellular component in the cell, for example, by a change in expression level or stability. Activity of a cellular component can also be modulated by a covalent modification of the molecule including, for example, addition of a phosphate by a kinase, removal of a phosphate by a phosphatase or addition or removal of other chemical moieties such as complex carbohydrates or hydrocarbons like prenyl, farnesyl, or geranylgeranyl groups. Further modulation of cellular component activity can include increase or decrease in activity due to a change in a level of a substrate or inhibitor of the component.

Delivery of electromagnetic energy in a method of the invention can modulate the activity of cellular components including, without limitation, cell cycle regulators, signal transduction proteins, transcription factors, DNA synthesis proteins or receptors. Examples of particular cellular components that can be activated or inhibited by delivery of electromagnetic energy in a method of the invention are set forth above in the definitions and in the Examples below. A regulatory network to which an electromagnetically affected component belongs will be influenced by the change in activity. Accordingly, a method of the invention can be used to modulate the activity of a network to which a particular component belongs. Those skilled in the art will know or be able to determine what networks are affected by a particular component and will be able to determine how the network is affected based on the change in activity of the component as it relates to its function in the network. One or more components that have modulated activity in response to delivery of electromagnetic energy can be used to monitor the effectiveness of treatment as set forth in further detail below.

Delivery of electromagnetic energy to a cell in a method of the invention can be used to activate mitogenic signaling pathways. As demonstrated in Example 1, electromagnetic energy stimulates release of soluble factors via transduction pathways that include ERK-1. The soluble factors themselves provide mitogenic stimuli to cells further activating mitogenic signaling pathways in a feed forward manner in the treated cells and additionally, stimulate mitogenic signaling pathways in other cells as well. Thus, delivery of electromagnetic energy to a cell can be used to modulate the activity of components in miotogenic signaling pathways including, for example, those set forth below.

Several different classes of kinases associated with mitogenic stimuli are known and are referred to as mitogen activated protein (MAP) kinases. MAP kinases can be classified into three types including extracellular signal regulated kinases (ERKs), c-Jun NH₂-terminal kinases (JNKs), and p38 kinases. The latter two are often grouped together as stress activated protein kinases (SAPKs). The two most predominant forms of ERK kinases are ERK-1 and ERK-2, also referred to as p44 and p42 MAP Kinases, respectively. They are ubiquitously expressed in the body, and, within cells, can be found in the cytoplasm, nucleus, and associated with the cytoskeleton. ERK-1 and ERK-2 are activated in fibroblasts by serum, growth factors, cytokines, and in some cases stress, although these pathways are typically considered non-stress pathways.

The JNK and p38 pathways are more traditionally associated with stress activation. JNKs can be activated by cytokines, agents that interfere with DNA and protein synthesis, or other stresses. They can also be activated by serum and growth factors, although less frequently. The p38 kinases are activated by cytokines, hormones, osmotic and heat shock, as well as other stresses.

While the JNK and p38 pathways are typically activated only by G-protein coupled receptors, the ERK pathway can be activated by both G-protein coupled receptors and tyrosine kinase receptors. The pathways activated by these two classes of receptors are distinct, but tend to overlap further down the cascade. As a result, activation of G-protein coupled receptors can result in activation of pathways associated with both classes of receptors.

G-protein coupled receptors are a broad group of receptors. They are involved in a wide variety of biological functions, including endocrine and exocrine regulation, exocytosis, platelet function, embryogenesis, angiogenesis, tissue regeneration, and control of cell growth.

Different G-protein coupled receptors can interact with the ERK's through several different pathways. In general, the cascade is activated when a ligand binds to the receptor, causing a conformational change in the α subunit of the G-protein. As a result of the conformational change, the α subunit exchanges a GDP for a GTP, thereby becoming active and liberating the βγ heterodimer. Both the α and the βγ subunits are able to activate ERK.

The diversity of interactions of the G-proteins with ERK pathways is primarily achieved through different varieties of α subunits. The Gα_{q} subunit interacts with the ERK pathway by controlling PLC-β, which hydrolizes phosphatidylinositol 4,5-biphosphate to form IP₃ and diacylglycerol (DAG), both of which are upstream effectors of ERK. In contrast, the Gαₛ subunit activates ERK by working through adenylyl cyclases, which generate cAMP, another upstream effector of ERK activation. Gα_{I}, on the other hand, inhibits activation of adenylyl cyclases. The activity of these and other α subunits involved in the ERK pathways can be modulated by delivery of electromagnetic energy to a cell in which they are expressed. The βγ heterodimer also plays a separate role in ERK activation, for example, in the JNK pathway, the βγ subunit, along with α₁₂ and α₁₃ are the G-protein subunits primarily responsible for activation.

Downstream of the G-proteins, there are several different factors that are independently activated, depending on the Gα subunit involved. One such set of factors, important in both the ERK and JNK pathways, are families of proteins known as GTPases. Associated with the ERK pathway is the Ras family of GTPases while associated with the JNK pathway is the Rho family. Ras is activated in response to the interactions of several proteins including, for example, Sos, a Ras-guanine nucleotide exchange factor; Grb2, an adapter protein; and Shc, which is activated by the βγ subunit. Activation of Ras results in formation of Ras-GTP and occurs when Sos associates with Grb2 and Shc, an interaction that occurs in association with tyrosine kinase receptors. Rho family proteins, including Rac1 and Cdc42, are activated by Gβγ, Gα₁₂, and Gα₁₃. Rac1 and Cdc42 in turn activate kinases upstream of JNK, such as PAK and MLK3/DLK.

As set forth above, the Gα_{q} subunit influences the activity of PLC-β, which cleaves phosphatidylinositol 4,5-biphosphate to form IP₃ and DAG. IP₃ and DAG work in concert to release intracellular stores of calcium and activate PKC. PKC activates Raf-1, a MAP Kinase Kinase Kinase, which also interacts with Ras.

The Gαₛ subunit activates the ERK pathway through interaction with adenylyl cyclases, of which there are at least 10 forms capable of generating cAMP. These forms of adenylyl cyclases are activated by Gαₛ, but they are differentially regulated by calcium, phosphorylation, βγ subunits and α inhibitory subunits. The changing concentrations of cAMP affect PKA activity in a cell-type dependent fashion. In fibroblasts and vascular smooth muscle cells, elevation of cAMP levels causes inhibition of ERK activation by interfering with PKA's ability to activate Raf-1. However, in ovarian, pituitary and neuronal cells, among others, elevation of cAMP levels promotes ERK activity by inactivating Raf-1 and stimulating PKA to activate Rap1 and B-Raf.

As set forth above, after the activation of G-proteins, activation can branch off in many different directions including, for example, along the Ras pathway, the PKA pathway, or the PKC pathway. These separate interactions are part of a greater network wherein the pathways influence each other, for example, through regulation of the activity of Rap1 and the Rafs. Rap1 is another GTPase, which, as set forth above, is activated by PKA. There are at least three forms of Raf including Raf-1, A-Raf, and B-Raf. Both B-Raf and Raf-1 can be activated by PKA, while only Raf-1 is activated by PKC. On the other hand, activation of Raf-1 by PKA can also be inhibited by high concentrations of cAMP, depending on cell type, as set forth above.

Interactions among Ras, Rap1 and the three forms of Raf influence multiple signal transduction pathways thereby acting as nodes connecting these pathways in a larger signal transduction network. Activation of all three Rafs requires the presence of active Ras, although only B-Raf can be activated solely by Ras. Rap1 can either stimulate or inhibit ERK activation. This is dependent on whether it is interacting with B-Raf, in which case it stimulates ERK activation, or Raf-1 and A-Raf, in which case it inhibits ERK activation.

The Rafs have MAP Kinase Kinase Kinase activity, and are referred to as MAPK/ERK Kinase Kinases (MEKKs). MEKKs typically act in conjunction with other proteins. For example, Raf-1 requires Ras and B-Raf requires Rap1. Other proteins also influence MEKK activity such as heat shock protein 90, p50, and 14-3-3. Acting with these other proteins, the Rafs are the major class of proteins responsible for the activation of the MEKs, which are immediately upstream of ERK. There are three forms of MEKs including MEK1a, MEK1b and MEK2 each of which specifically activates ERKs. The JNK family has a separate set of activating kinases known as SKK1/SEK1, which are activated independently of the Rafs.

All of the ERKs are activated by dual phosphorylation on an activation loop that contains a threonine and tyrosine separated by a glutamate. The tyrosine is phosphorylated first. The ERKs with a single phosphorylation accumulate in the cell to a threshold level, above which they are converted to the fully active, dual-phosphorylated form. After activation, an ERK translocates to the nucleus, where it modulates the activity of a number of transcription factors involved with the regulation of normal and aberrant cell growth, including c-Myc, Elk-1 and ATF2. An ERK can also interact with other factors involved in DNA and protein synthesis including, for example, other kinases, such as Rsk2, which phosphorylates histone H3; MAP Kinase interacting kinases (Mnk) 1 and 2, which are responsible for activating eukaryotic initiation factor 4E (eIF-4E), which initiates protein synthesis; heat shock factor transcription factor 1 (hsp1); and topisomerase II-b, among others. The JNKs activate transcription factors as well including, for example, c-Jun, Elk-1, Elk-2, ATF2 and serum response factor accessory protein (Sap-1). The p38 kinases also activate ATF2 and Elk.

A method of the invention can be used to deliver an effective amount of electromagnetic energy to modulate a component of a mitogenic signal transduction pathway set forth above. Modulation of the activity of a component in a signal transduction pathway can lead to changes in the activity of other components in the pathway or in a related pathway in accordance with the molecular interactions set forth above as well as others known in the art as described, for example, in Houslay et al., Molecular Pharmacology 58:659-668 (2000); Lopez-Ilasaca, Biochemical Pharmacology 56:269-277 (1998); Marinissen et al., Sciences 22:368-376 (2001); and Pearson et al., Endocrine Reviews 22:153-183 (2001).

Changes in the activity or level of a cellular component can be correlated with other effects of delivery of electromagnetic energy such that changes in the activity or level of a cellular component can be monitored to determine the effectiveness of electromagnetic therapy. Accordingly, the invention provides a method for monitoring progress of electromagnetic therapy, by detecting a level of a cellular component in a cell population following delivery of electromagnetic energy to the cell population, whereby the level of the cellular component correlates with the effectiveness of the therapy. The method can be used with any cellular component that changes activity or level in response to electromagnetic energy such as those set forth above and in the Examples.

The progress of electromagnetic therapy can be monitored based on the activity or level of a single gene product or a plurality of gene products. The level or activity of a gene product can be determined using methods well known in the art such as mRNA detection methods and protein detection methods described, for example, in Sambrook et al., Molecular Cloning: A Laboratory Manual, 2nd ed., Cold Spring Harbor Press, Plainview, N.Y. (1989); Sambrook et al., Molecular Cloning: A Laboratory Manual, 3rd ed., Cold Spring Harbor Press, Plainview, N.Y. (2001) or Ausubel et al. (Current Protocols in Molecular Biology (Supplement 47), John Wiley & Sons, New York (1999)). Additionally, activity of gene products can be measured using known enzyme assays such as kinase assays or binding assays that exploit interactions and activities such as those described above in regard to particular gene products.

Monitoring a plurality of gene products provides the advantage of being able to determine the effects that the treatment has upon a signal transduction pathway or a network of interacting pathways. Examples of methods known in the art for measuring the levels of a plurality of gene products include cDNA sequencing, clone hybridization, differential display, subtractive hybridization, cDNA fragment fingerprinting, serial analysis of gene expression (SAGE), and mRNA or protein microarrays. Example II describes the use of micro-array analysis for determining changes in expression for plurality of gene products in response to the delivery of electromagnetic energy. Methods of detecting the activity or level of one or more gene products can be performed either qualitatively or quantitatively.

Based on the activity or level of a cellular component determined in a diagnostic method of the invention, a course of therapy can be modified. In this regard, the invention provides a method for modifying electromagnetic therapy. The method includes the steps of: (a) detecting a level of a cellular component in a cell population following delivery of electromagnetic energy to the cell population, whereby the level of the cellular component correlates with the growth of the cell population, and (b) modifying the electromagnetic therapy based on the level of the cellular component in the cell population.

One or more of the cellular components described herein can be detected in a method of modifying electromagnetic therapy. The effective dose of electromagnetic energy can be reduced or increased depending upon the particular cellular component detected and its level. In the case where a cellular component is detected to be above a desired level, the effective dose of electromagnetic energy can be reduced. On the other hand, if a particular cellular component is detected to be below a desired level, the effective dose of electromagnetic therapy can be increased. The desired level of one or more cellular components can be determined based on a correlation with desired outcomes in a model system or patient population in a clinical setting or using other correlation analyses known in the art. Electromagnetic therapy can be modified by altering one or more of the parameters described above such that the effective amount of electromagnetic energy delivered to the cell or tissue being treated is either increased or decreased.

The molecular processes regulating the main events of the cell cycle are similar in all eucaryotic cells. Thus, an effective amount of electromagnetic energy when delivered to any eukaryotic cell in a method of the invention can be used to accelerate its cell cycle. Examples of cells that are useful in a method of the invention are described below in the context of particular applications of the invention such as wound healing in which the cell cycle is accelerated for stromal cells, fibroblasts, keratinocytes, neutrophils, epitheleal cells or macrophages; healing of neuronal damage in which the cell cycle of neuronal cells and glia is accelerated; and production of artificial tissues in which-the cell cycle is accelerated for fibroblasts, smooth muscle cells, endothelial cells, plasma cells, mast cells, macrophages/monocytes, adipocytes, pericytes reticular cells found in bone marrow stroma, or chondrocytes.

Electromagnetic energy can be delivered to a cell in vitro or in vivo using a method of the invention. A cell that is treated in vitro using a method of the invention can be a primary cell or tissue sample obtained directly from an individual. A cell or tissue can be readily obtained using minimally invasive methods, for example, from fluids such as the blood or lymph or from accessible tissues such as the skin, hair follicles, cervix or cheek. Where necessary a cell can also be obtained using slightly more invasive procedures, such as a punch biopsy, needle biopsy, endoscope biopsy or surgical biopsy. Depending on the need and the availability of an appropriate procedure, cells from essentially any organ or tissue of the body can be obtained for use in a method of the invention. Those skilled in the art will know or be able to determine an appropriate method for obtaining a cell of interest based on various factors including, for example, the location of the cell and risk factors or preference of the individual from whom the cell is harvested.

A cell used in an in vitro embodiment of the invention can be further isolated from other biological components. For example, a cell that is treated with electromagnetic energy can be a primary cell disaggregated from connective tissue and irrelevant cells using, for example, known methods such as enzymatic digestion and biochemical separation. Likewise, a cell used in a method of the invention can be separated from other cells, for example, using affinity separation methods known in the art. As an example, flow cytometry, selective media or antibody panning methods can be used to select a population of cells expressing a detectable surface marker. Thus, a cell used in a method of the invention can be a single isolated cell or a cell in a population of cells such as a biological fluid, tissue or organ. A cell whether isolated or in a tissue or other population can be propagated in culture for several generations, if desired. Cells can be propagated using methods known in the art as described, for example, in Freshney, Culture of Animal Cells, 4th Ed. Wiley-Liss, New York (2000).

Delivery of electromagnetic energy to cells in vitro can be used to increase the rate at which the culture propagates. Thus, the methods can be used to decrease the cost and time required to obtain a cell culture-that has grown to a desired density or to a point of acquiring other favorable characteristics. A culture that has been stimulated to proliferate in vitro in a method of the invention can be used in an in vitro application. Examples of in vitro applications for which a cell treated in a method of the invention can be used include diagnostic methods, cell based drug screening methods or biofermentation methods for production of biological agents. Alternatively, a cell or population of cells that has been stimulated to proliferate in vitro can be subsequently administered to an individual in an in vivo therapeutic method. For example, the methods can be used to stimulate formation of a tissue in vitro under conditions in which a replacement tissue or organ is formed. Once formed or grown to an appropriate stage, a tissue or organ can be administered to an individual in need of the tissue or organ. The use of electromagnetic energy to stimulate tissue formation in vitro as well as in vivo is described in further detail below.

Electromagnetic energy can be delivered directly to a cell or to the environment of the cell such as a culture medium, tissue, fluid or organ in which the cell is located. For in vivo applications of the method, electromagnetic radiation can be delivered directly to a site to be treated or to a location that is sufficiently proximal that the target cell will be electromagnetically affected. As an example, electromagnetic energy can be delivered externally to treat conditions or diseases that afflict cells of the skin or that afflict internally located cells that are electromagnetically affected by surface application of electromagnetic energy. Alternatively, electromagnetic energy can be delivered to an internal site by surgical exposure of the site or endoscopic access to the site.

A cell used in a method of the invention can be genetically manipulated, for example, to include an exogenous nucleic acid. Thus, a method of the invention can include a step of introducing an exogenous nucleic acid into a cell to which electromagnetic energy is delivered. An exogenous nucleic acid can be introduced into a cell using well known methods of transduction or transfection as described, for example, in Freshney et al., supra (2000); Sambrook et al., Molecular Cloning: A Laboratory Manual, 2nd ed., Cold Spring Harbor Press, Plainview, N.Y. (1989); Sambrook et al., Molecular Cloning: A Laboratory Manual, 3rd ed., Cold Spring Harbor Press, Plainview, N.Y. (2001) or Ausubel et al. (Current Protocols in Molecular Biology (Supplement 47), John Wiley & Sons, New York (1999)). An exogenous nucleic acid can be introduced into a cell in order to provide a diagnostic capability to the cell. Exemplary exogenous nucleic acids that can provide a diagnostic capability to a cell include, without limitation, those that express reporter genes such as Green Fluorescent Protein (GFP), and wavelength shifted variants thereof; chloramphenicol acetyltransferase; beta-galactosidase; beta-glucuronidase; or luciferase. An exogenous nucleic acid that is introduced into a cell can also express a therapeutic gene product such as a growth factor, hormone, or blood clotting factor. A therapeutic gene product can be expressed in vitro and subsequently delivered to an individual in need of the gene product in a pharmaceutical formulation or a cell expressing a therapeutic gene product can be introduced into an individual in need of the gene product in order to treat a disease or condition. Similarly, an exogenous nucleic acid encoding other gene products that are useful in the manufacture or production of therapeutics, foods, or industrial chemicals can be produced in vitro from a cell containing the nucleic acid. Electromagnetic energy can be delivered to a cell that contains an exogenous nucleic acid prior to expression of the nucleic acid or concurrently with its expression.

The invention also provides a method for reducing inflammation. The method includes a step of delivering to a tissue undergoing inflammation an effective amount of electromagnetic energy to reduce the inflammation. A collection of immune system cells and molecules at a target site is known in the art as inflammation, a common response to injury or infection that is identified by four classic symptoms including heat (calor), redness (rubor), swelling (tumor) and pain (dolor). Acute inflammatory response, which is induced by antibodies or other agents, is characterized by a set of rapidly occurring events at the site of injury. Vessels located near the site of the injury dilate, thereby causing redness and heat, allowing an influx of plasma proteins and phagocytic cells into the tissue spaces, thereby causing swelling. Release and/or activation of other inflammation mediators, and increased tissue pressure, stimulate local nerve endings, causing pain. The methods of the invention can be used to reduce or ameliorate symptoms associated with inflammation. Delivery of electromagnetic energy in the methods leads to reduction in inflammation by promoting reduction in inflammatory processes occurring in the cells set forth below, thereby allowing progression to subsequent healing stages.

In an infection, if the acute inflammatory response relieves the host of the infectious agent, repair and regeneration ensue. However, if the acute response is not effective in ridding the host of the infection, the continued influx of polymorphonuclear leukocytes and serum products can lead to formation of abscesses and granulomas. The abscess is a swelling which is bounded by fibrin from clotted blood and cells involved in phagocytosis and repair. The central cavity of the abscess contains both live and dead polymorphonuclear leukocytes, tissue debris, and the remaining injurious or infecting agent.

A continuing acute inflammatory response can lead to a chronic inflammatory response, which is associated with the same four clinical signs described above, but is composed of additional cellular and soluble mediators. Chronic inflammatory responses are characterized by an infiltration of lymphocytes and cells of monocyte-macrophage lineage in addition to polymorphonuclear cells.

Both acute and chronic inflammation include three phases. In the first phase, the material to be eliminated (antigen) is recognized as foreign by various mechanisms involving immunoglobulins. Following recognition, the second phase of the immune response is initiated, during which an amplification system involving complement, cytokines, kinins, coagulation, lipid mediators, and a large number of inflammatory cells is activated. This results in an alteration of blood flow, increased vascular permeability, augmented adherence of circulating leukocytes to the vascular endothelium, promotion of migration of leukocytes into tissue, and stimulation of leukocytes to destroy the inciting antigen. During the third phase, destruction of the antigen is mediated by several non-specific mechanisms including phagocytic cells such as neutrophils, eosinophils and mononuclear phagocytes. Such phagocytic leukocytes migrate freely or are fixed in tissue sites as components of the mononuclear phagocyte system.

The first immune cells to arrive at the site of inflammation are neutrophils, generally within a few hours of tissue injury or infection. Neutrophils are produced in the bone marrow and take approximately two weeks to achieve maturity. The first seven days of neutrophil development are proliferative, and with successive cell division the cells evolve from myeloblasts to promyelocytes and then to myelocytes. During this period neutrophils acquire their characteristic granules. The first granules to appear during neutrophil maturation are called the primary or azurophil granules. Primary granules function predominantly in the intercellular environment, in the phagolysosomal vacuole where they are involved in killing and degrading microorganisms.

Macrophages perform a similar function to neutrophils as well as more diverse tasks. These ubiquitous and mobile cells continually sample their environment and respond to various stimuli. Macrophages are highly active in absorptive endocytosis or pinocytosis, and in receptor-mediated endocytosis. When particles are internalized by these processes, antimicrobial and general cytotoxic activity is promoted, thereby killing infectious agents.

Wound repair is an example of a healing process that is characterized by an initial inflammatory response followed by later stages of healing. In particular, the natural course of wound healing to closure occurs in four phases identified as acute inflammation, granulation, epithelialization and tissue remodeling. During the inflammation phase there is an immigration of neutrophils into the area of injury within the first 24 hours. Within the subsequent 24 to 48 hours, the immunocyte profile changes as the infiltrate begins to consist predominantly of macrophages and lymphocytes. In another 24 to 48 hours, macrophages and lymphocytes become the predominant cell types within wound tissue. It is also during the inflammatory phase that monocytes are converted to macrophages, which release growth factors for stimulating angiogenesis and the production of fibroblasts. In one embodiment of the invention, wound repair is accelerated by delivering to the wound an effective amount of electromagnetic energy to reduce inflammation at the wound.

In further embodiments the methods of the invention can be used to deliver electromagnetic energy to other tissues undergoing inflammation to reduce the inflammation and promote healing. Examples of tissues that can be treated in a method of the invention when undergoing inflammation include, without limitation, neural tissue associated with a neuroinflammatory disorder, gastrointestinal tissue associated with an inflammatory bowel disorder or ulcer, synovium tissue associated with arthritic inflammation, lung tissue associated with asthma, or skin associated with an inflammatory skin condition such as psoriasis, eczema or atopic dermatitis. The cell or tissue to which electromagnetic energy is delivered in a method of the invention can be one that is not associated with a wound. Thus, although the methods are exemplified herein with respect to wounds, the methods can be used to treat inflammation associated with a disease or condition other than a wound.

As described in further detail below, the methods of the invention are useful for reducing both acute and chronic inflammation. For example, the methods of the invention are useful for reducing acute inflammation associated with, for example, swelling resulting from bumps (contusions), bruises, sprains, abrasions, cuts, insect stings, plant-induced contact dermatitis as can be caused by plants such as poison ivy, poison oak or poison sumac.

The methods of the invention also are useful for reducing the severity of a neuroinflammatory disorder, for example, a demyelinating disease. A central mechanism in the pathology of neuroinflammatory demyelinating diseases is the organ-specific migration of activated T lymphocytes into the brain. Additionally, injury to the spinal cord precipitates the activation of resident microglia and the recruitment of circulating inflammatory cells, including macrophages and lymphocytes. These cells can cause tissue damage and loss of neurological function via autoimmune reactions to myelin proteins. Autoimmunity can be trauma-induced leading to ongoing central nervous system (CNS) immunologic responses by the autoreactive repertoire. Accordingly, the methods of the invention are applicable in the context of CNS trauma and neurodegenerative diseases such as for example, Multiple Sclerosis (MS), Chronic Inflammatory Demyelinating Polyneuropathy, Amyotrophic Laterial Sclerosis (ALS) and Alzheimer's Disease.

Demyelinating diseases are an important group of neurological disorders because of the frequency with which they occur and the disability that they cause. Demyelinating diseases have in common a focal or patchy destruction of myelin sheaths that is accompanied by a neuroinflammatory response. Neuroinflammatory demyelinating diseases can be divided into processes affecting myelin of the central nervous system and those affecting myelin of the peripheral nervous system. Multiple Sclerosis (MS) is a central nervous system demyelinating disease with an autoimmune etiology as reviewed in Martin et al., Annu. Rev. Immunol. 10:153-187 (1992). Other demyelinating diseases of the central nervous system include, for example, acute disseminated encephalomyelitis (ADE) including postinfectious and postvaccinal encephalomyelitis, acute necrotizing hemorrhagic encephalomyelitis and progressive (necrotizing) myelopathy. Demyelinating diseases of the peripheral nervous system include, for example, acute inflammatory demyelinating polyradiculoneuropathy (Guillain-Barre syndrome), chronic inflammatory demyelinating polyradiculoneuropathy (CIDP), demyelinating neuropathy associated with IgM monoclonal gammopathy and neuropathy associated with sclerosing myeloma. A method of the invention can be used to treat a neuroinflammatory disease or condition by delivering to a neural tissue undergoing inflammation an effective amount of electromagnetic energy to reduce the inflammation.

The methods of the present invention are further useful for reducing inflammation associated with Crohn's disease (CD) and ulcerative colitis (UC), two gastrointestinal disorders that are collectively referred to as Inflammatory Bowel Disease (IBD); or regional enteritis, which is a disease of chronic inflammation that can involve any part of the gastrointestinal tract, by delivering an effective amount of electromagnetic energy to a cell or tissue at a site undergoing inflammation associated with these disorders. Commonly the distal portion of the small intestine (ileum) and cecum are affected. In other cases, the disease is confined to the small intestine, colon or anorectal region. Crohn's disease occasionally involves the duodenum and stomach, and more rarely the esophagus and oral cavity.

Several features are characteristic of the pathology of Crohn's disease. The inflammation associated with CD, known as transmural inflammation, involves all layers of the bowel wall. Thickening and edema, for example, typically appear throughout the bowel wall, with fibrosis also present in long-standing disease. The inflammation characteristic of CD also is discontinuous with segments of inflamed tissue, known as “skip lesions,” separated by apparently normal intestine. Furthermore, linear ulcerations, edema, and inflammation of the intervening tissue lead to a “cobblestone” appearance of the intestinal mucosa, which is distinctive of CD.

A hallmark of Crohn's disease is the presence of discrete aggregations of inflammatory cells, known as granulomas, which are generally found in the submucosa. About half of Crohn's disease cases display the typical discrete granulomas, while others show a diffuse granulomatous reaction or nonspecific transmural inflammation. As a result, the presence of discrete granulomas is indicative of CD, although the absence granulomas also is consistent with the disease. Thus, transmural or discontinuous inflammation, rather than the presence of granulomas, is a preferred diagnostic indicator of Crohn's disease (Rubin and Farber, Pathology (Second Edition) Philadelphia: J.B. Lippincott Company (1994)).

The methods of the present invention are also useful for reducing inflammation associated with ulcerative colitis by delivering an effective amount of electromagnetic energy to a cell or tissue at a site affected by UC. Several pathologic features characterize UC in distinction to other inflammatory bowel diseases. Ulcerative colitis is a diffuse disease that usually extends from the most distal part of the rectum for a variable distance proximally. The term left-sided colitis describes an inflammation that involves the distal portion of the colon, extending as far as the splenic flexure. Sparing of the rectum or involvement of the right side (proximal portion) of the colon alone is unusual in ulcerative colitis. Furthermore, the inflammatory process of UC is limited to the colon and does not involve, for example, the small intestine, stomach or esophagus. In addition, ulcerative colitis is distinguished by a superficial inflammation of the mucosa that generally spares the deeper layers of the bowel wall. Crypt abscesses, in which degenerate intestinal crypts are filled with neutrophils, also are typical of the pathology of ulcerative colitis (Rubin and Farber, Pathology (Second Edition) Philadelphia: J.B. Lippincott Company (1994), which is incorporated herein by reference).

A characteristic endoscopic feature of UC, which when present with clinical features of left-sided colonic disease indicates ulcerative colitis, is inflammation that is more severe distally than proximally or continuous inflammation. Additional typical endoscopic features that may be present in UC include inflammation extending proximally from the rectum or shallow ulcerations or the lack of deep ulcerations.

A method of the invention can also be useful for reducing inflammation occurring at a joint, for example, associated with arthritis. An example of a joint disease is rheumatoid arthritis (RA) which involves inflammatory changes in the synovial membranes and articular structures as well as muscle atrophy and rarefaction of the bones, most commonly the small joints of the hands. Inflammation and thickening of the joint lining, called the synovium, can cause pain, stiffness, swelling, warmth, and redness. The affected joint may also lose its shape, resulting in loss of normal movement and, if uncontrolled, may cause destruction of the bones, deformity and, eventually, disability. In some individuals, RA can also affect other parts of the body, including the blood, lungs, skin and heart. A method of the invention can be useful for reducing one or more of these adverse symptoms by reducing inflammation associated with RA.

A method of the invention can be used to replace damaged tissue by treating the damaged tissue with an effective amount of electromagnetic energy to stimulate growth of a replacement tissue. Examples of tissues that can be replaced in a method of the invention include, without limitation, epithelial tissue, bone marrow tissue, smooth muscle tissue, connective tissue, adrenal tissue and neurological tissue. A tissue that is replaced in a method of the invention can be located anywhere in the body that is accessible to delivery of electromagnetic energy including, for example, in a blood vessel, vein, artery, tendon, ligament, gastrointestinal tract, genitourinary tract, bone marrow, skin, liver, pancreas, lung, kidney, or nervous system including the central or peripheral nervous system. Because the inability to restore and preserve normal tissue structure after damage is a major cause of organ failure, such as failure of the liver, kidney, or heart, the methods of the invention are particularly useful for reducing the risk of organ failure.

A replacement tissue that is induced to proliferate by delivery of electromagnetic energy in a method of the invention can be derived from native cells that are naturally occurring in the individual being treated. Examples of cells that are capable of replacing skin cells include those described above with respect to wound healing. Cells that can be stimulated in a method of the invention to replace a tissue of the circulatory system include, for example, fibroblasts, smooth muscle cells, and endothelial cells. The methods can be used to simultaneously stimulate proliferation of distinct cell types such as fibroblasts, smooth muscle cells, capillary cells or lymphocytes that are useful for replacing tissues of the gastrointestinal tract. Stimulation of fibroblasts is also useful for replacing tendons and ligaments. Parenchymal cells and other known tissue specific cells can be used to replace damaged portions of organs. Damaged tissue of the nervous system can be replaced with neurons or glia cells. It will be understood that replacement of the cell types set forth above by stimulating their proliferation includes stimulation of precursor cells and stem cells that differentiate into the cell types set forth above.

The invention further provides a method for stimulating growth of administered cells. The method includes the steps of (a) administering a population of cells to an individual, and (b) delivering to the population an effective amount of electromagnetic energy to stimulate growth of the population. In one embodiment, the population of cells can be administered to a site of tissue damage, such as those described above, and stimulated to replace the damaged tissue. A population of cells can also be administered in a method of treating other defects in the body such as the deficiency or over abundance of a particular gene product. Accordingly, a method of the invention can include administering cells that either naturally express an effective amount of a gene product for a desired therapeutic effect or that have been genetically manipulated to do so using, for example, the methods described above.

A cell or population of cells administered to an individual in a method of the invention can be any type of cell that is appropriate for replacing a tissue or performing a desired function including, for example, those set forth above. A population of cells that is administered in a method of the invention can be in a tissue or organ that is isolated as a tissue or organ from a donor individual or that is produced in a culture system. Methods for producing synthetic tissues or organs are set forth in further detail below.

For therapeutic applications, the above cell types are additionally chosen to remain viable in vivo without being substantially rejected by the host immune system. Therefore, the donor origin of the cell type should be evaluated when selecting cells for therapeutic administration. A cell can be autologous, wherein it is administered to the same individual from whom it was removed or can be heterologous being obtained from a donor individual who is different from the recipient individual. Those skilled in the art know what characteristics should be exhibited by cells to remain viable following administration. Moreover, methods well known in the art are available to augment the viability of cells following administration into a recipient individual.

One characteristic of a donor cell type substantial immunological compatibility with the recipient individual. A cell is immunologically compatible if it is either histocompatible with recipient host antigens or if it exhibits sufficient similarity in cell surface antigens so as not to elicit an effective host anti-graft immune response. Specific examples of immunologically compatible cells include autologous cells isolated from the recipient individual and allogeneic cells which have substantially matched major histocompatibility (MHC) or transplantation antigens with the recipient individual. Immunological compatibility can be determined by antigen typing using methods well known in the art. Using such methods, those skilled in the art will know or can determine what level of antigen similarity is necessary for a cell or cell population to be immunologically compatible with a recipient individual. Tolerable differences between a donor cell and a recipient can vary with different tissues and can be readily determined by those skilled in the art.

In addition to selecting cells which exhibit characteristics that maintain viability following administration to a recipient individual, methods well known in the art can be used to reduce the severity of immunorejection. Such methods can be used to further increase the in vivo viability of immunologically compatible cells or to allow the in vivo viability of less than perfectly matched cells or of non-immunologically compatible cells. Therefore, for therapeutic applications, it is not necessary to select a cell type from the recipient individual to achieve viability of the modified cell following administration. Instead, and as described further below, alternative methods can be employed which can be used in conjunction with essentially any donor cell to confer sufficient viability of the modified cells to achieve a particular therapeutic effect.

For example, in the case of partially matched or non-matched cells, immunosuppressive agents can be administered to render the host immune system tolerable to administration of the cells. The regimen and type of immunosuppressive agent to be administered will depend on the degree of MHC similarity between the donor cell and the recipient. Those skilled in the art know, or can determine, what level of histocompatibility between donor and recipient antigens is applicable for use with one or more immunosuppressive agents. Following standard clinical protocols, administration and dosing of such immunosuppressive agents can be adjusted to improve viability of the cells in vivo. Specific examples of immunosuppressive agents useful for reducing a host immune response include, for example, cyclosporin, corticosteroids, and the immunosuppressive antibody known in the art as OKT3.

Another method which can be used to confer sufficient viability of partially-matched or non-matched cells is through the masking of the cells or of one or more MHC antigen(s) to protect the cells from host immune surveillance. Such methods allow the use of non-autologous cells in an individual. Methods for masking cells or MHC molecules are well known in the art and include, for example, physically protecting or concealing the cells, as well as disguising them, from host immune surveillance. Physically protecting the cells can be achieved, for example, by encapsulating the cells within a barrier device. Alternatively, antigens can be disguised by treating them with binding molecules such as antibodies that mask surface antigens and prevent recognition by the immune system.

Immunologically naive cells also can be administered in a method of the invention. Immunologically naive cells are devoid of MHC antigens that are recognized by a host immune system. Alternatively, such cells can contain one or more antigens in a non-recognizable form or can contain modified antigens that mirror a broad spectrum of MHC antigens and are therefore recognized as self-antigens by most MHC molecules. The use of immunologically naive cells therefore has the added advantage of circumventing the use of the above-described immunosuppressive methods for augmenting or conferring immunocompatibility onto partially or non-matched cells. As with autologous or allogeneic cells, such immunosuppressive methods can nevertheless be used in conjunction with immunologically naive cells to facilitate viability of the administered cells.

An immunologically naive cell, or broad spectrum donor cell, can be obtained from a variety of undifferentiated tissue sources, as well as from immunologically privileged tissues. Undifferentiated tissue sources include, for example, cells obtained from embryonic and fetal tissues. An additional source of immunologically naive cells include stem cells and lineage-specific progenitor cells. These cells are capable of further differentiation to give rise to multiple different cell types. Stem cells can be obtained from embryonic, fetal and adult tissues using methods well known to those skilled in the art. Such cells can be used directly or modified further to enhance their donor spectrum of activity.

Immunologically privileged tissue sources include those tissues which express, for example, alternative MHC antigens or immunosuppressive molecules. A specific example of alternative MHC antigens are those expressed by placental cells, which prevent maternal anti-fetal immune responses. Additionally, placental cells are also known to express local immuno-suppressive molecules that inhibit the activity of maternal immune cells.

An immunologically naive cell or other donor cell can be modified to express genes encoding, for example, alternative MHC or immuno-suppressive molecules that confer immune evasive characteristics. Such a broad spectrum donor cell, or similarly, any of the donor cells described previously, can be tested for immunological compatibility by determining its immunogenicity in the presence of recipient immune cells. Methods for determining immunogenicity and criteria for compatibility are well known in the art and include, for example, a mixed lymphocyte reaction, a chromium release assay or a natural killer cell assay. Immunogenicity can be assessed by culturing donor cells together with lympohocyte effector cells obtained from a recipient individual and measuring the survival of the donor cell targets. The extent of survival of the donor cells is indicative of, and correlates with, the viability of the cells following implantation.

The invention further provides a method for stimulating formation of a tissue. The method includes the steps of: (a) contacting a population of cells with a matrix under conditions suitable for tissue formation by the cells, and (b) delivering to the population an effective amount of electromagnetic energy to stimulate formation of the tissue. One or both of the steps of the method can be carried out either in vitro or in vivo.

A matrix can be used in a method of the invention to provide a structural scaffold for a tissue. Such a scaffold can provide a substrate to which a tissue is adhered thereby localizing the tissue to a particular location in the body. If desired, the matrix can further be shaped to produce a tissue with a desired morphology. Examples of materials that are particularly useful as matrices include, without limitation, nylon (polyamides), dacron (polyesters), polystyrene, polypropylene, polyacrylates, polyvinyl compounds (e.g., polyvinylchloride), polycarbonate (PVC), polytetrafluorethylene (PTFE; teflon), thermanox (TPX), nitrocellulose, polyglycolic acid (PGA), cotton, cat gut sutures, cellulose, gelatin, dextran or an in vivo site such as bone, other tissues or a wound.

A matrix can be pre-treated prior to inoculation of cells in order to enhance the attachment of the cells to the matrix. For example, prior to inoculation with cells, nylon matrices can be treated with 0.1M acetic acid, and incubated in polylysine, FBS, and/or collagen to coat the nylon. Polystyrene can be similarly treated using sulfuric acid.

Where the matrix and cells are to be implanted in vivo, it may be preferable to use biodegradable matrices such as polyglycolic acid, catgut suture material, or gelatin, for example. Where the cultures are to be maintained for long periods of time or cryopreserved, non-degradable materials such as nylon, dacron, polystyrene, polyacrylates, polyvinyls, teflons or cotton can be used. A convenient nylon mesh that can be used in accordance with the invention is Nitex, a nylon filtration mesh having an average pore size of 210 microns and an average nylon fiber diameter of 90 microns (#3-210/36, Tetko, Inc., N.Y.).

Conditions suitable for in vivo formation of a tissue when a population of cells is contacted with a matrix include those described above in regard to replacement of damaged tissues except that the matrix is provided in a manner that does not interfere with the process of tissue replacement. In general, the matrix is provided under sterile conditions to avoid infection of the damaged tissue site. The matrix is further disposed in an orientation that allows cells to adhere to the matrix and, if desired, migrate along the matrix to form the tissue. The methods described above for delivering electromagnetic energy to a population of cells for replacing damaged tissue can be used in the presence of a matrix as well.

Conditions suitable for in vitro formation of a tissue when a population of cells is contacted with a matrix are known in the art and described, for example, in U.S. Pat. Nos. 5,842,477; 5,863,531; 5,902,741 and 5,962,325. An in vitro tissue can be cultured in a closed system bioreactor as described, for example, in U.S. Pat. No. 6,121,042. The methods and apparatus known in the art can be modified to deliver electromagnetic energy to a cell culture system. Those skilled in the art will be able to make such modifications by providing an electromagnetic energy delivery device to the culture system according to the teachings described herein.

A method of the invention for stimulating formation of a tissue in vitro can further include a step of administering the tissue to an individual. Such a tissue can be administered using methods described above with regard to administering cells to wounds and other sites of tissue damage. Those skilled in the art will know or be able to determine placement of a synthetic tissue based on structural properties inherent in the tissue such as morphology and cell composition. Cell types and tissues capable of the in vivo or in vitro formation methods of the invention include, for example, macrophages, neutrophils, fibroblasts, muscle cells, epithelial cells, keratinocytes, microvascular and other endothelial cells, epidermal melanocytes, hair follicle papilla cells, skeletal muscle cells, smooth muscle cells, osteoblasts, neurons, chondrocytes, hepatocytes, pancreatic cells, kidney cells, aortic cells, bronchial/tracheal cells (both epithelial and muscle cells).

The following examples are intended to illustrate but not limit the present invention.

### EXAMPLE I

### Induction of Cell Proliferation with Electromagnetic Energy

This example demonstrates delivery of electromagnetic energy to cells in vitro and activation of extracellular signal-regulated protein kinase 1 (ERK-1 or p44 kinase) and other components associated with mitogenic signaling pathways.

Primary Human Dermal Fibroblasts (HDF) and Human Epidermal Keratinocytes (HEK)(Cell Applications, Inc., San Diego Calif.) were used between passages 3 and 15 and 3-8, respectively. Unless stated otherwise, all cell culture supplies were purchased from Mediatech Inc. (Herdon, Va.). Minimum Essential Medium (MEM) was used for culture of the HDFs. This medium was supplemented with 5% fetal bovine serum (Hyclone, Logan, Utah), 1 mM sodium pyruvate, 100 U/ml penicillin G, 100 U/ml streptomycin and 1% non-essential amino acids. Serum-free growth media (Cell Applications, Inc. San Diego Calif.) was used for culturing the HEK cells.

The primary HDF cells were synchronized, then treated with radio frequency energy (RF) as follows. Briefly, cells plated at 1000 cells/well in 96 well plates were synchronized using Compactin as described in Keyomarsi et al., Cancer Res. 51:3602-3609 (1991) with the following modifications. Media was removed 30-36 hours after and the cells were then treated with a 100× excess of Mevalonolactone (MAL) to release them from Compactin synchronization. 30 minutes after MAL treatment the cells were treated with a 30 minute dose of RF at 32 mw/cm².

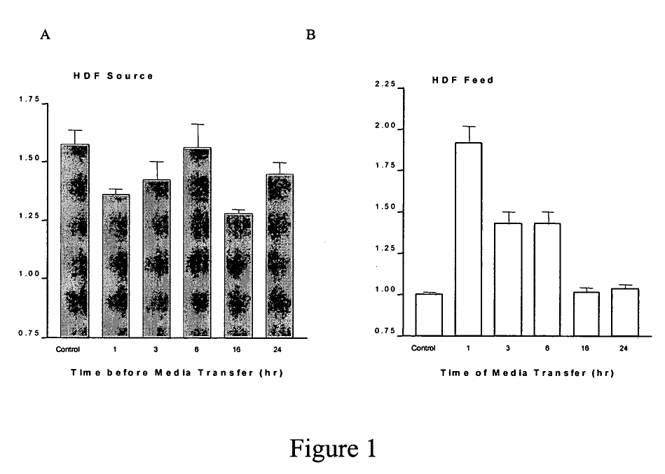

As shown in FIG. 1, treatment of HDF cells with RF induced proliferation. Furthermore, untreated cells showed induced proliferation when media from the RF treated cells was added within 1 hour. For the media transfer analysis, HDF cells in 5% FBS-MEM were plated at 2.5×10³ cells/well in 96 well plates and allowed to attach overnight. Plates were treated with RF consisting of a dose of 32 mw/cm² provided as a train of 42 μsec pulses delivered at a rate of 1 Khz for 30 minutes. Media was then removed after 1 hour, 3 hours, 6 hours, 16 hours, 24 hours after RF treatment and placed on untreated cells. All plates were assayed for cell growth twenty-four hours after RF treatment. Cell growth was quantified using CyQUANT™ Cell Proliferation Assay Kit (Molecular Probes, Eugene, Oreg.). Comparison of the stimulation of cell growth by RF treatment was similar to treatment of cells with known growth factors such as platelet derived growth factor (PDGF), further indicating that RF treatment of cells stimulates cell growth in a similar fashion as endogenous growth factors.

In addition to inducing proliferation in the HDF cells, RF treatment induced the concomitant activation of the ERK signaling pathway. Levels of ERK were detected as follows. Treated cells were washed once with cold phosphate-buffered saline, lysed in Laemmli sample buffer (Bio-Rad, Hercules, Calif.) and sonicated. Samples were heated to 95° C., electrophoresed on 12.5% SDS gels and transferred to PVDF membrane (Osmonics, Inc., Westborough, Mass.) by semi-dry transfer in CAPS buffer (pH 11). The PVDF membrane was blocked in Tris-buffered saline (20 mM Tris-HCl, 130 mM NaCl, pH 7.6) containing 5% non-fat dry milk. The membrane was then incubated with anti-phosphorylated p44/42 MAP Kinase (Thr202/Tyr204) antibody or anti-p44/42 antibody (Cell Signaling Technology, Beverly, Mass.) overnight at 4° C., and washed in Tris-buffered saline supplemented with 1% Tween-20 (AP Biotech, Piscataway, N.J.). The membrane was visualized with the Amersham ECF kit (Piscataway, N.J.) according to the manufacturers protocol and imaged on a Storm 840 PhosphoImager.

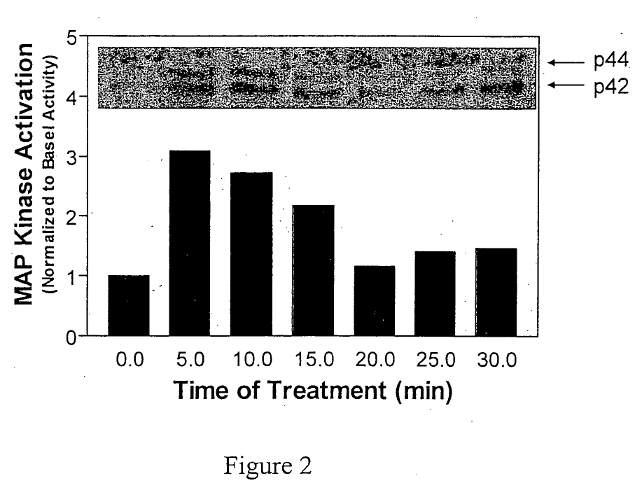

Using the above-described Western blotting techniques, cell lysates were probed for the presence of activated ERK-1 and ERK-2 at specific times after initiation of treatment. As shown in FIG. 2, significant activation of ERK-1 and ERK-2, resulting in 250% increased levels compared to control cells occurred after the initiation of treatment. ERK activation within the first 30 minutes of treatment indicated that RF activated this Kinase cascade in a biologically relevant time frame for affecting cellular functions such as cell cycle progression and cell proliferation.

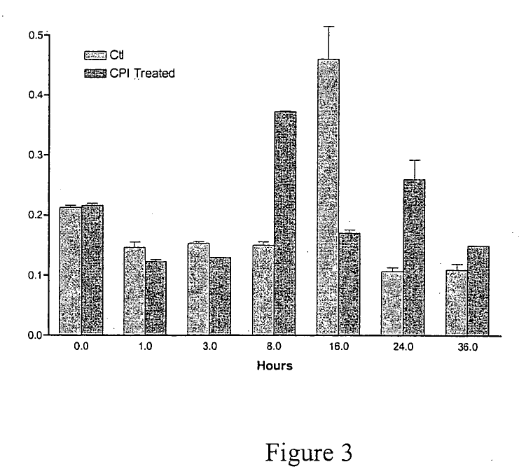

To determine if RF treatment increased the rate of entry into the S phase of the cell cycle, DNA synthesis was imaged in the synchronized HDF cells following treatment with RF. Entry into the S phase of the cell cycle was determined with the Roche (Mannheim, Germany) BrdU labeling and detection kit. Briefly, cultured cells were labeled with BrdU, fixed with ethanol, and incubated with nucleases to partially digest cellular DNA. Anti-BrdU antibody conjugated to peroxidase was incubated with the cells. Peroxidase substrate was added to the plates producing a colored product that was measured with an ELISA reader. As shown in FIG. 3, the RF treated cells entered the S phase of the cell cycle on average, 8 hours before untreated controls.

These results demonstrate that RF acts as an exogenous, non-molecular mitogen. The results further demonstrate that RF induces the release of soluble factors via a transduction pathway that includes ERK-1, and that the resulting soluble factor release re-stimulates the mitogenic signaling pathway as demonstrated by the second phase of ERK-1 activation.

### EXAMPLE II

### Activation of Molecular Regulatory Networks with Electromagnetic Energy

This example demonstrates delivery of electromagnetic energy to cells leading to modulation in the levels of gene products associated with molecular regulatory networks. The levels of various components are shown to be modulated within the first few minutes to several hours following delivery of electromagnetic energy.

HDF cells were cultured in MEM supplemented with 5% fetal calf serum as described in Example I. Cells were plated in 10 cm plates at a density of 5×10⁵ cells per plate. Twenty hours after plating electromagnetic energy was delivered to the cells as described in Example I. RNA was harvested from cells at various times according to the method of Chomczynski, P. and Sacchi, Analytical Biochemistry 162 pg. 156-159 (1987). Fifty μg of total RNA was treated with DNAse I for 30 minutes followed by phenol extraction and ethanol precipitation. The RNA was then labeled with ³²P dATP using reverse transcriptase. Labeled probes were then purified by column chromatography and then hybridized to micro-arrays at 1×10⁵ cpm/ml at 68° C. for 24 hours. Micro-arrays were purchased from BD Biosciences Clontech (Palo Alto, Calif.). Two arrays were used: the Atlas array 1.2 (1,174 cDNA clones) and a Stress array which contained 234 cDNA clones of genes related to cellular stress. The list of the genes and their sequences can be found on the world wide web at the website for BD Biosciences Clontech. The blots were washed and then exposed for 5-7 days at −80° C. using double intensifying screens. Quantitation of transcript levels between blots was performed by normalization to housekeeping gene expression.

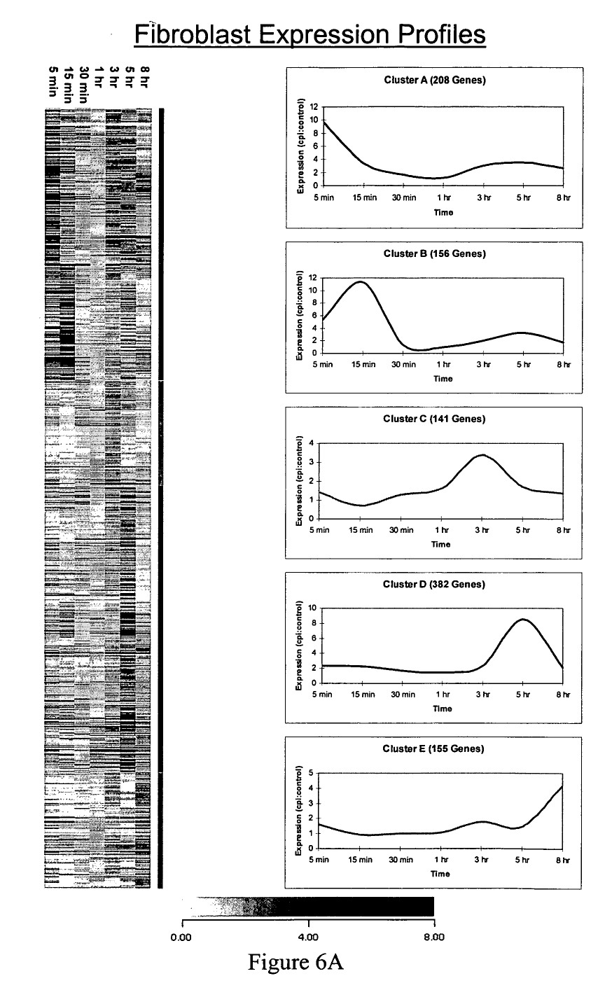

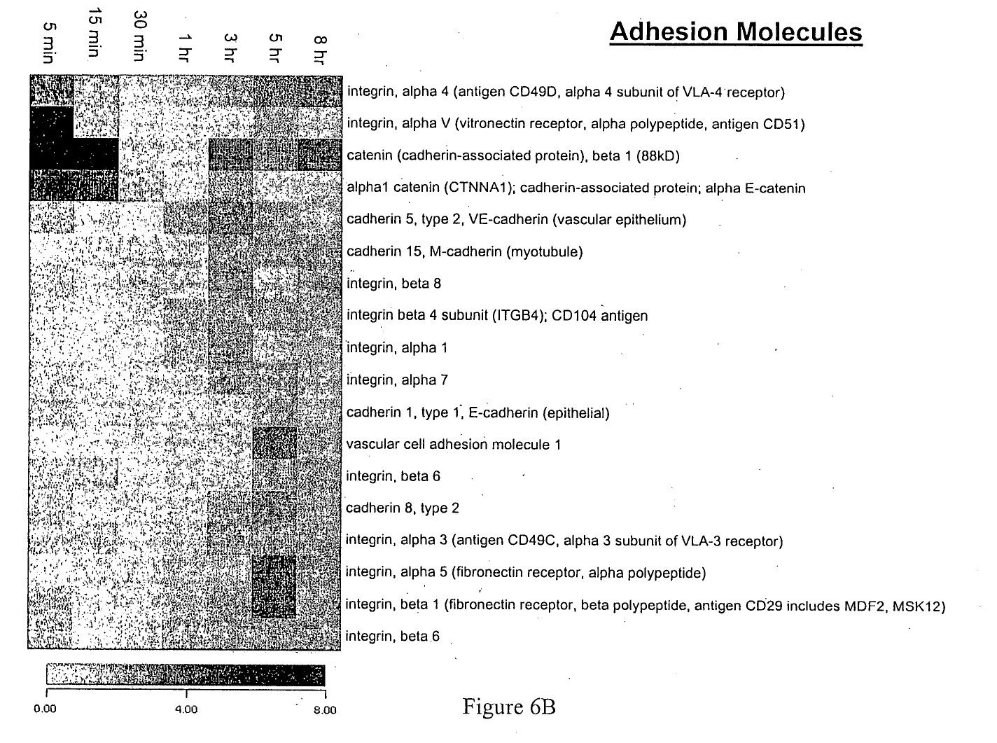

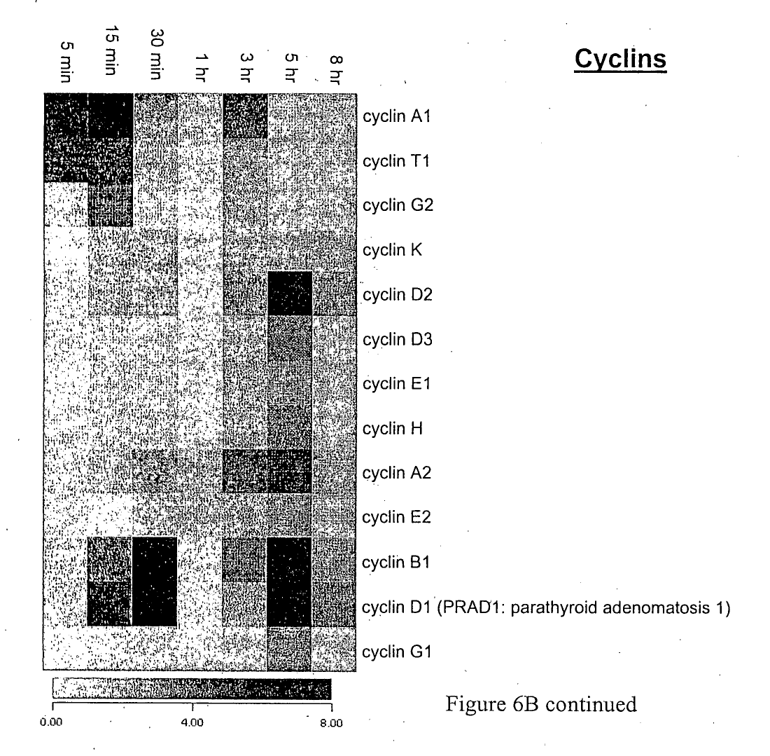

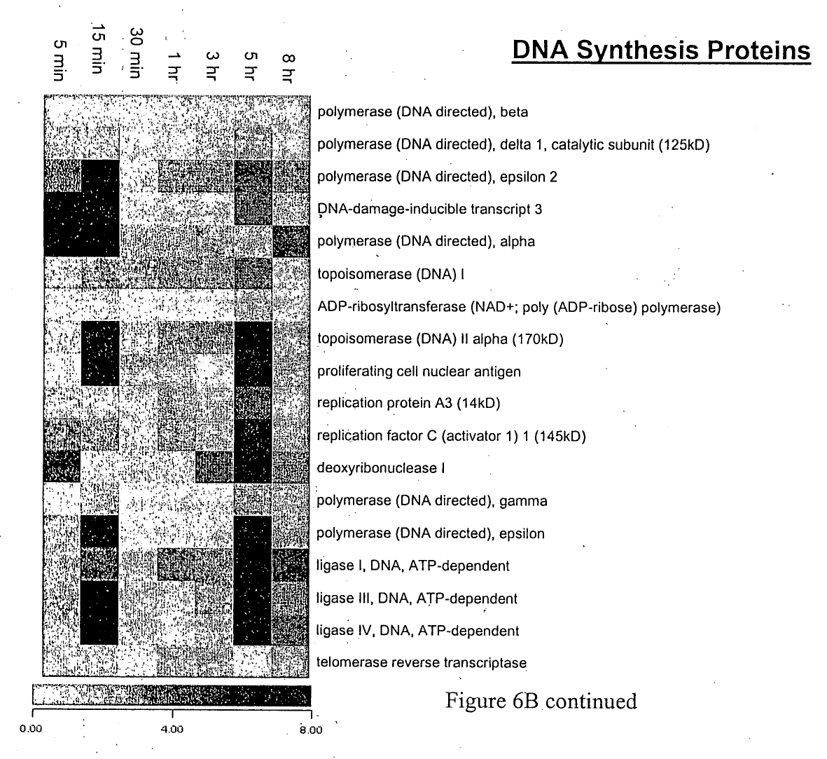

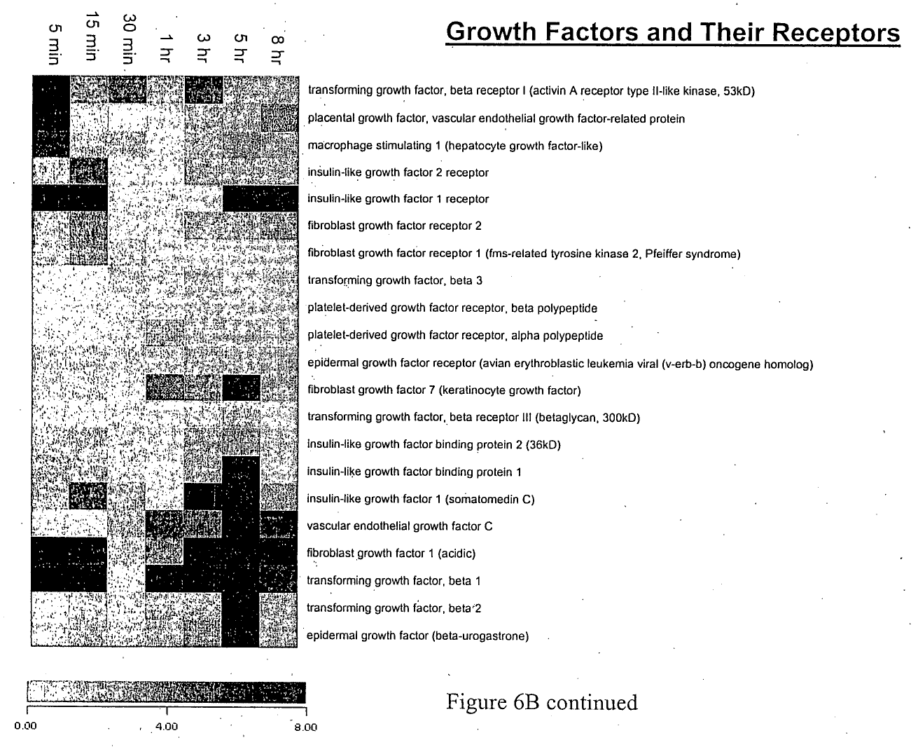

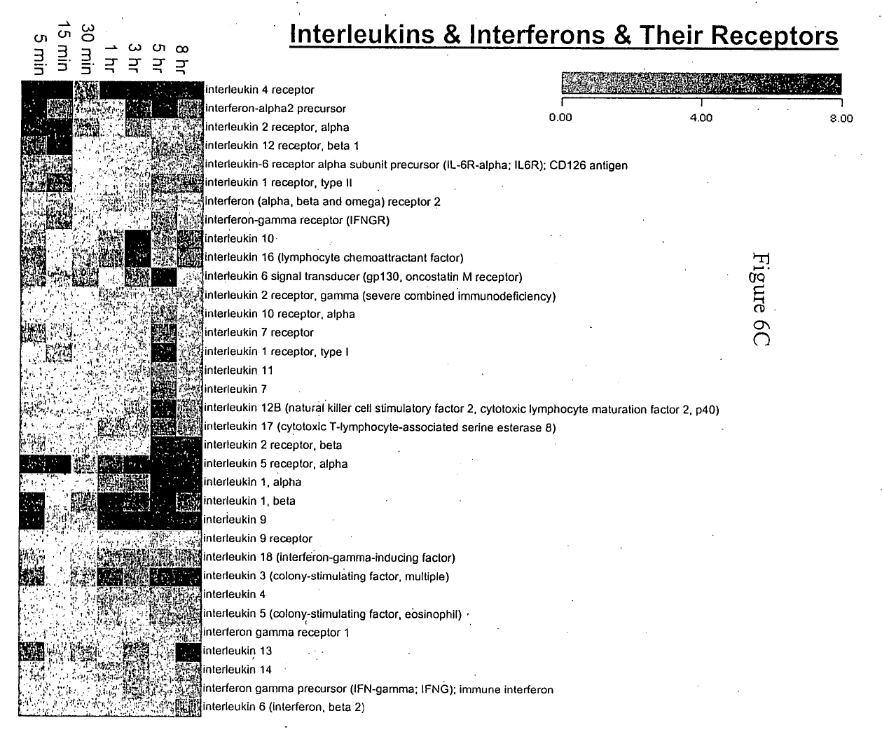

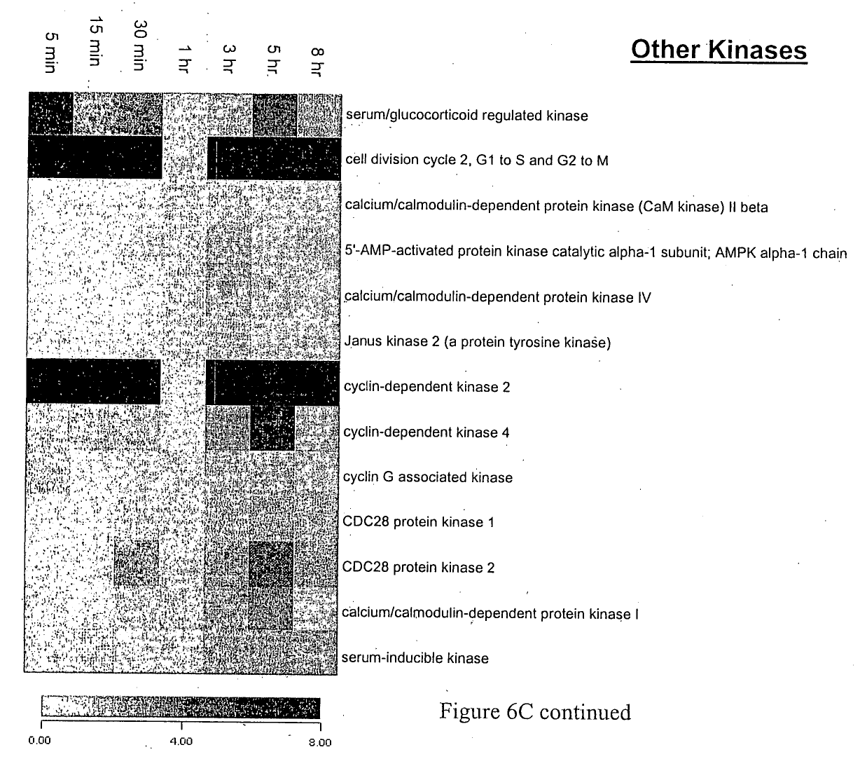

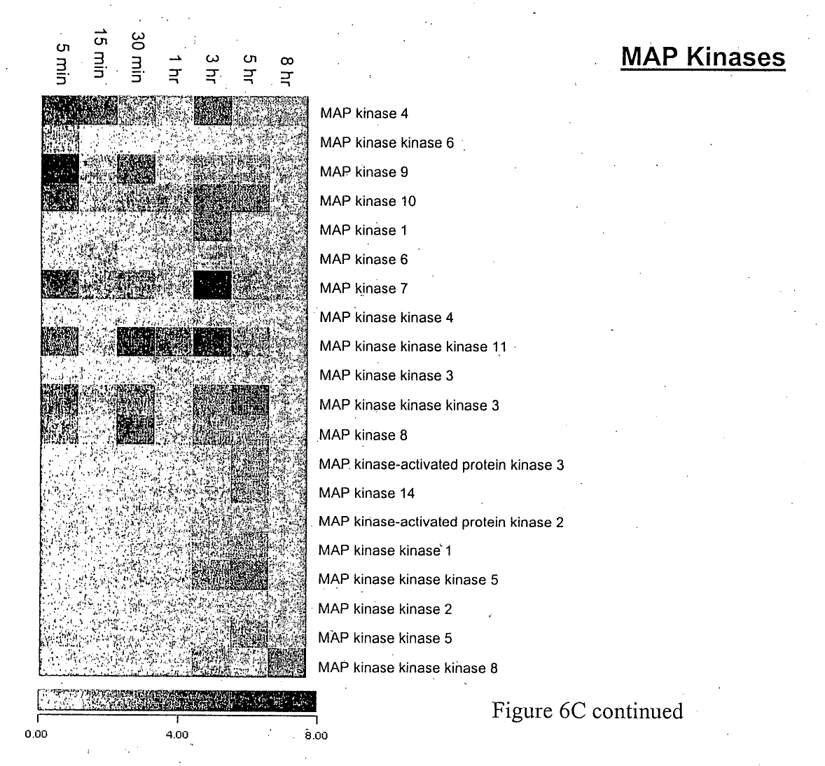

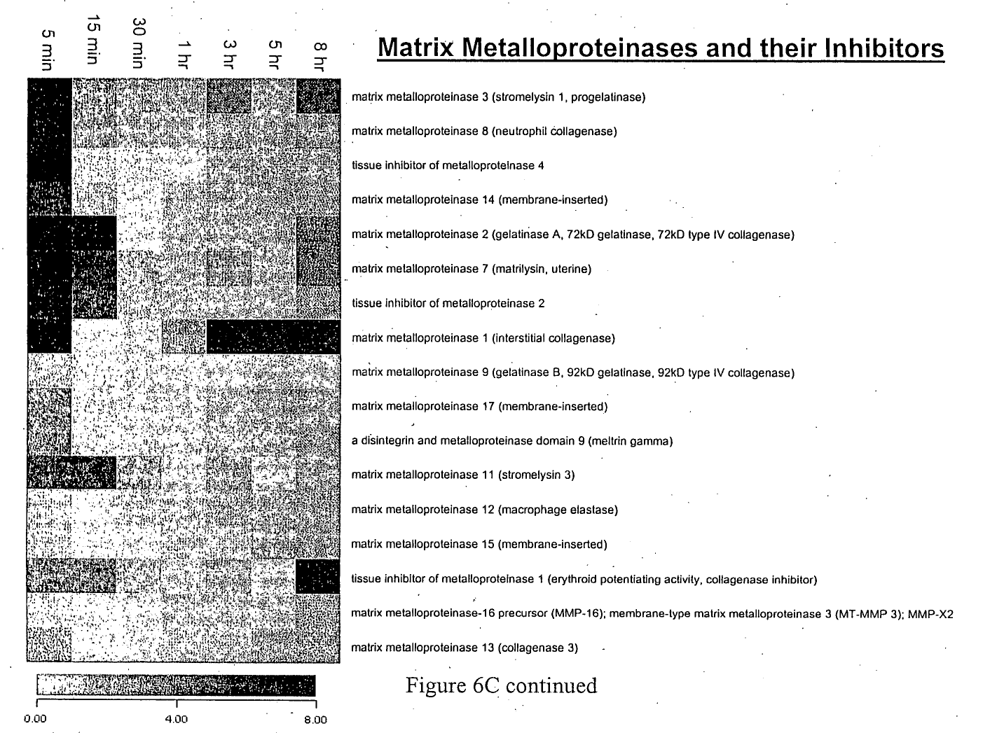

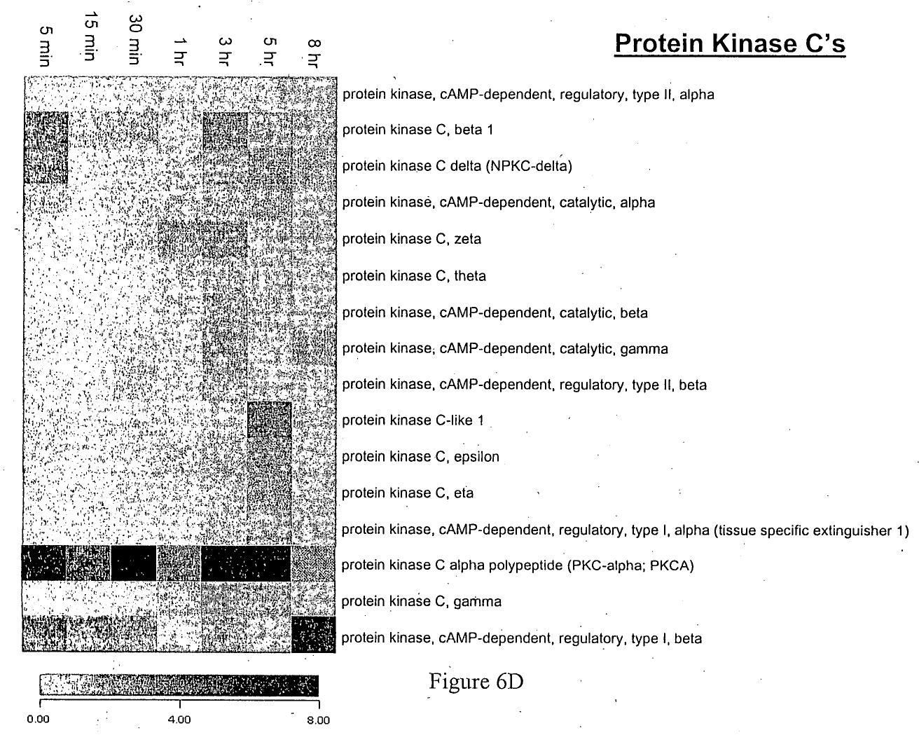

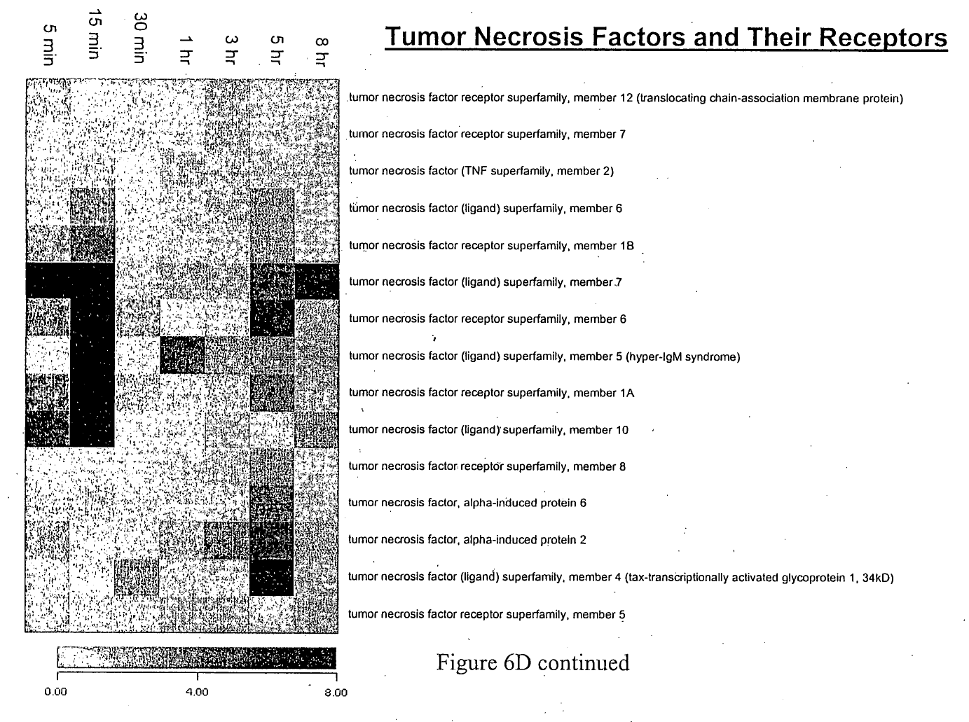

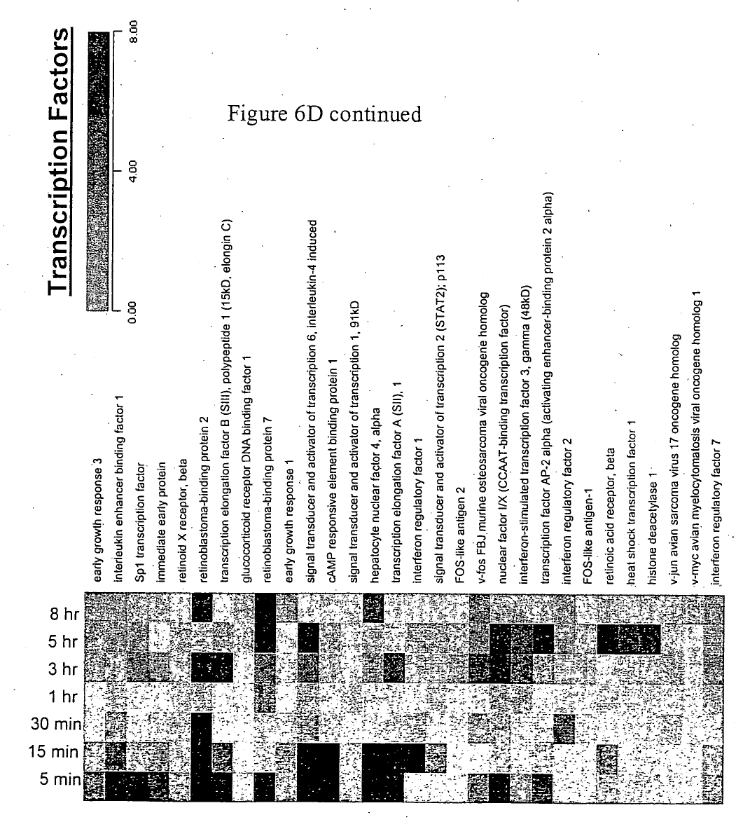

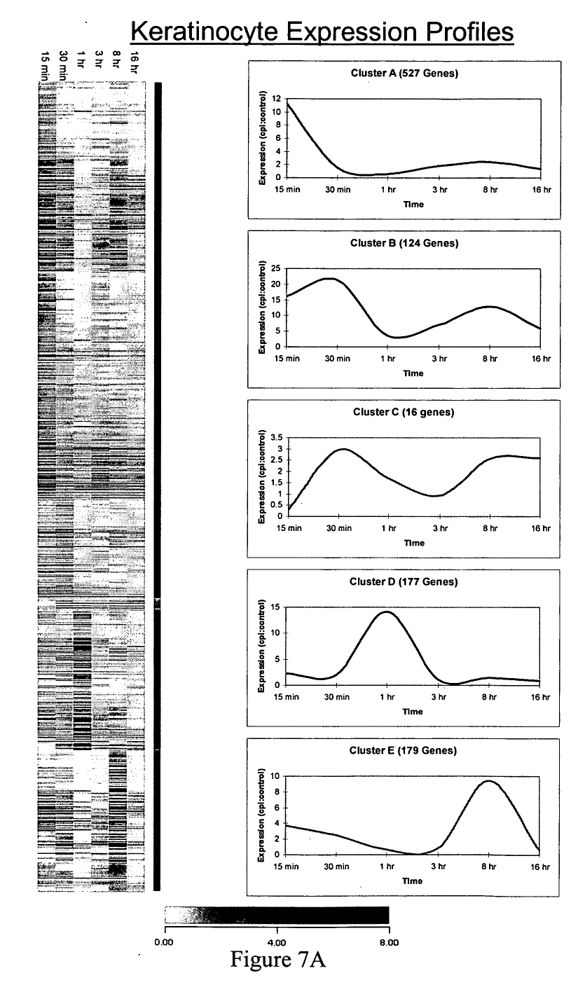

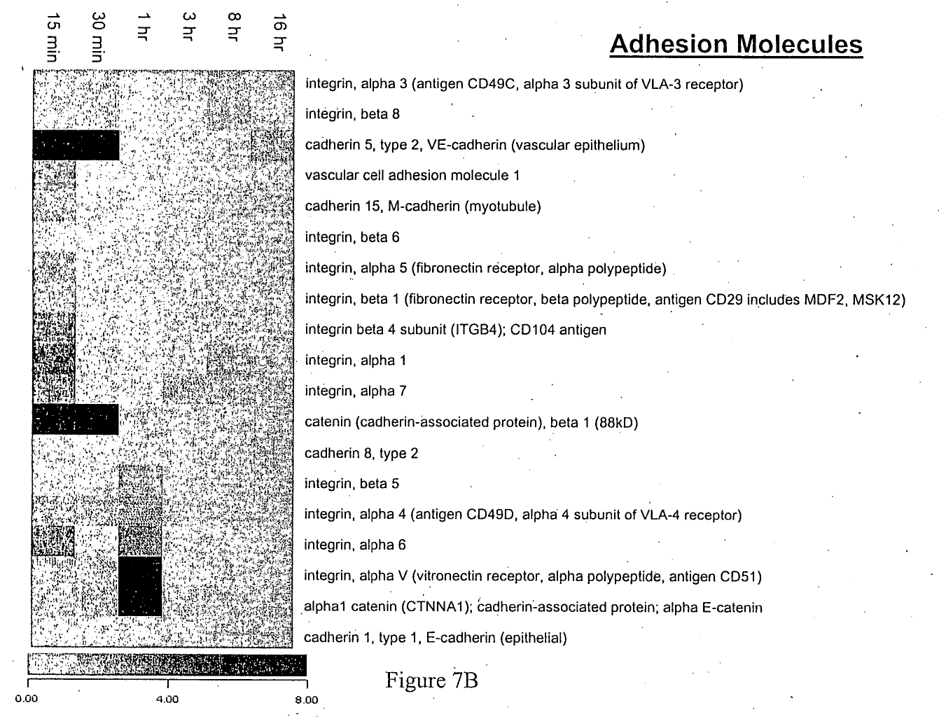

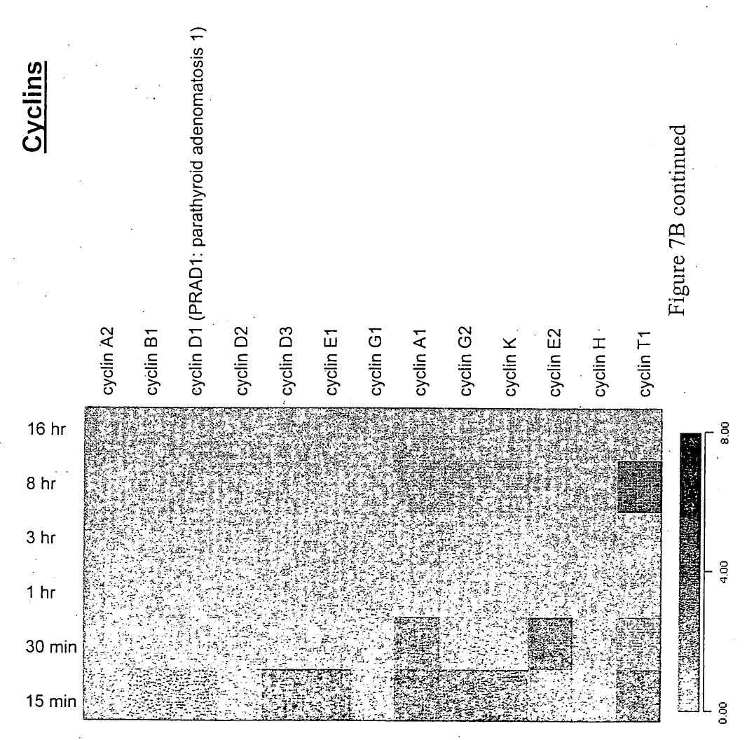

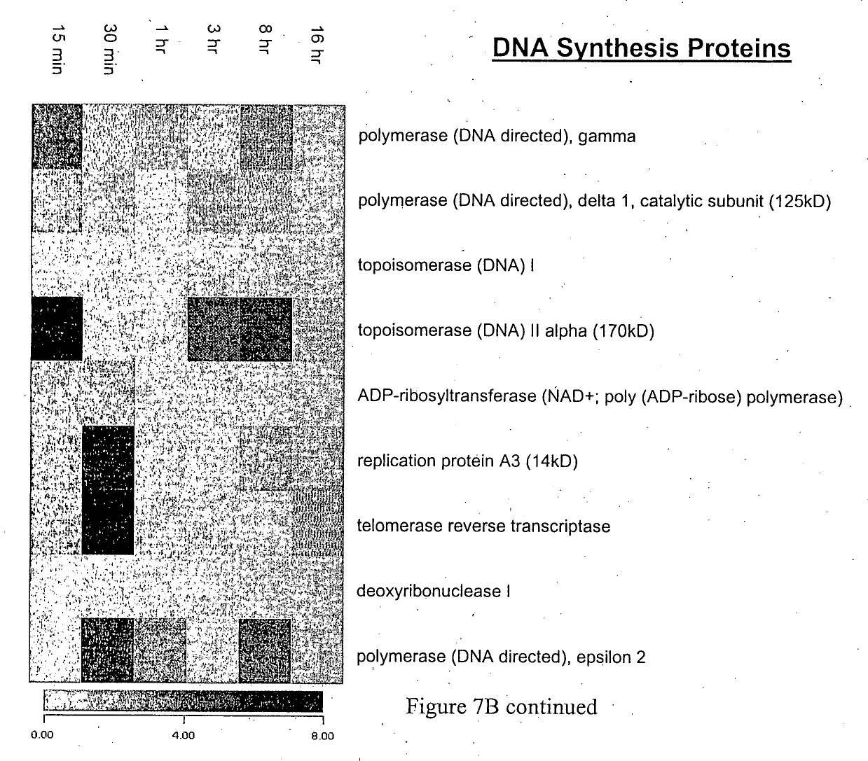

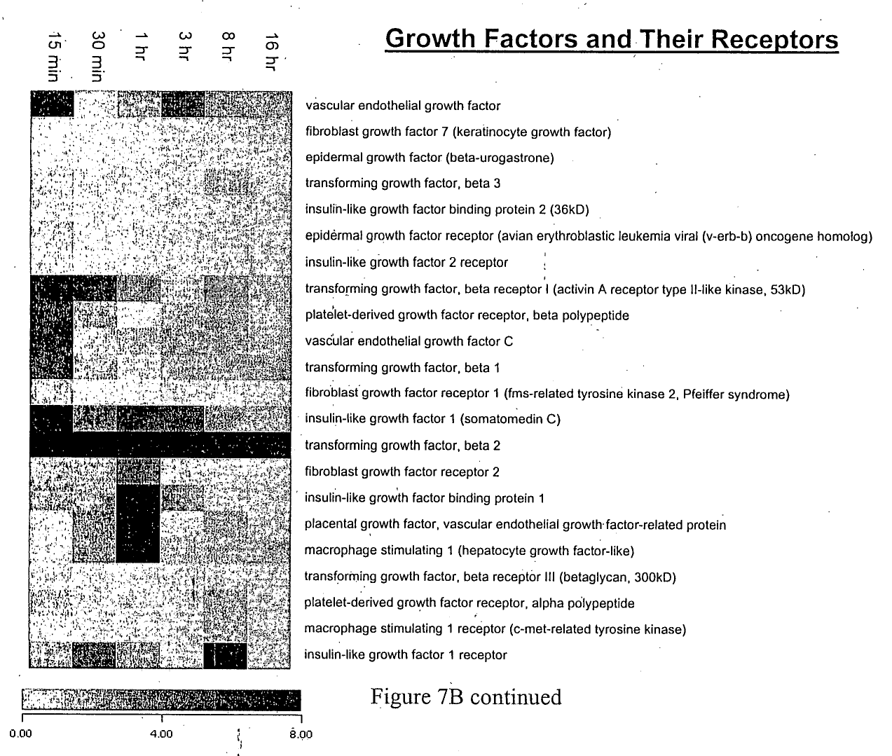

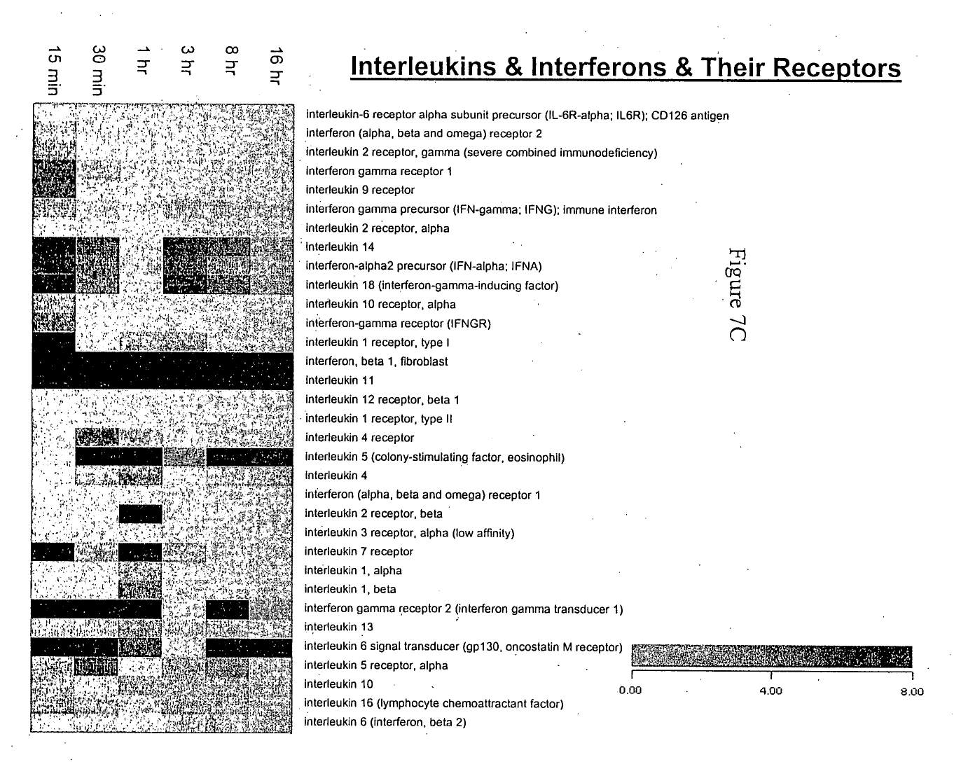

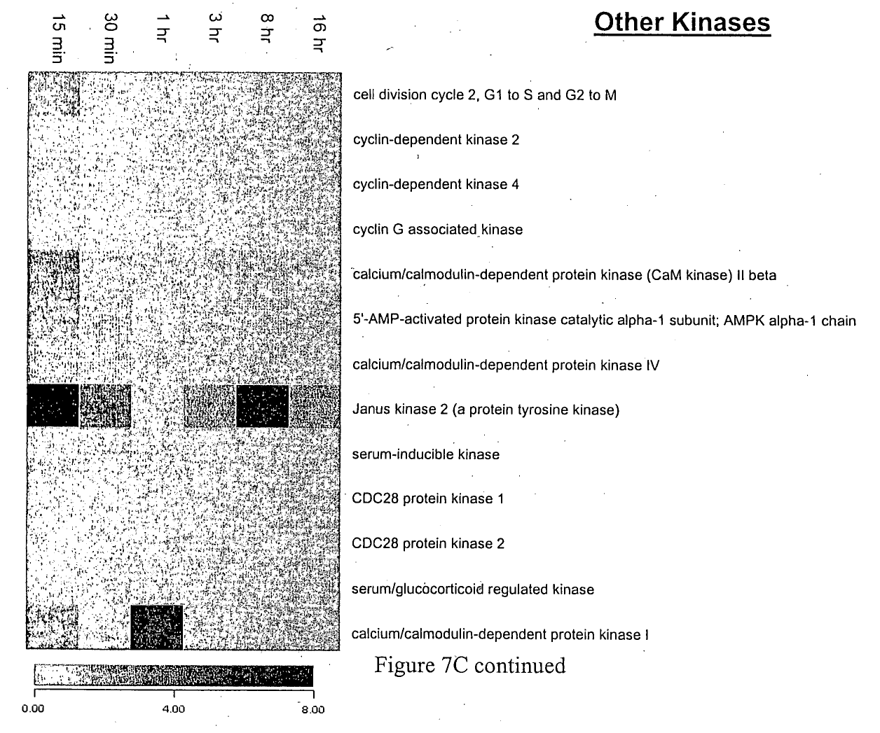

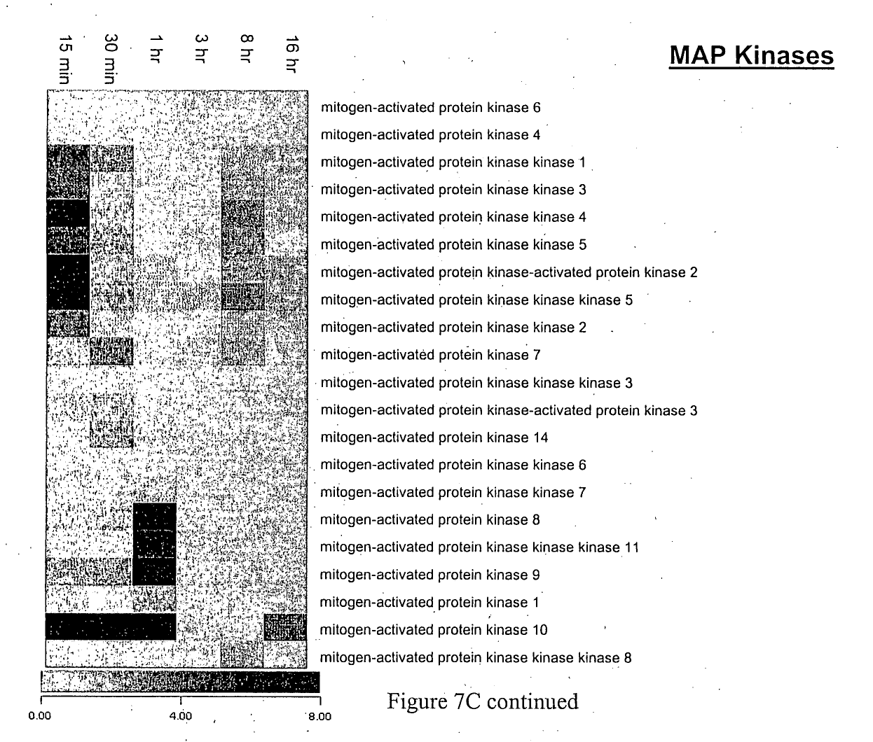

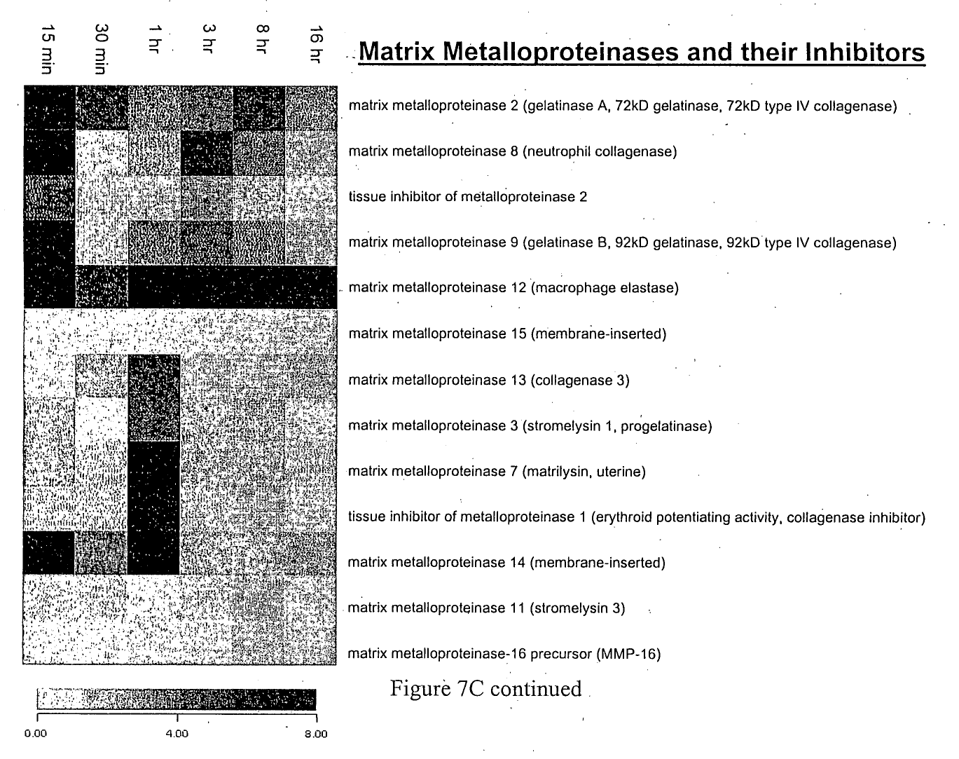

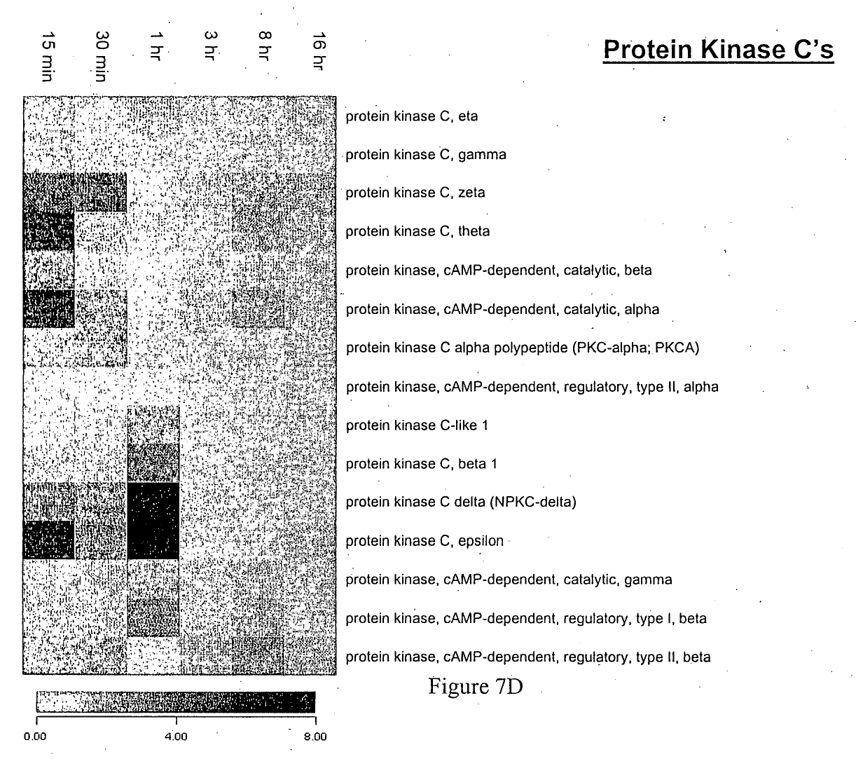

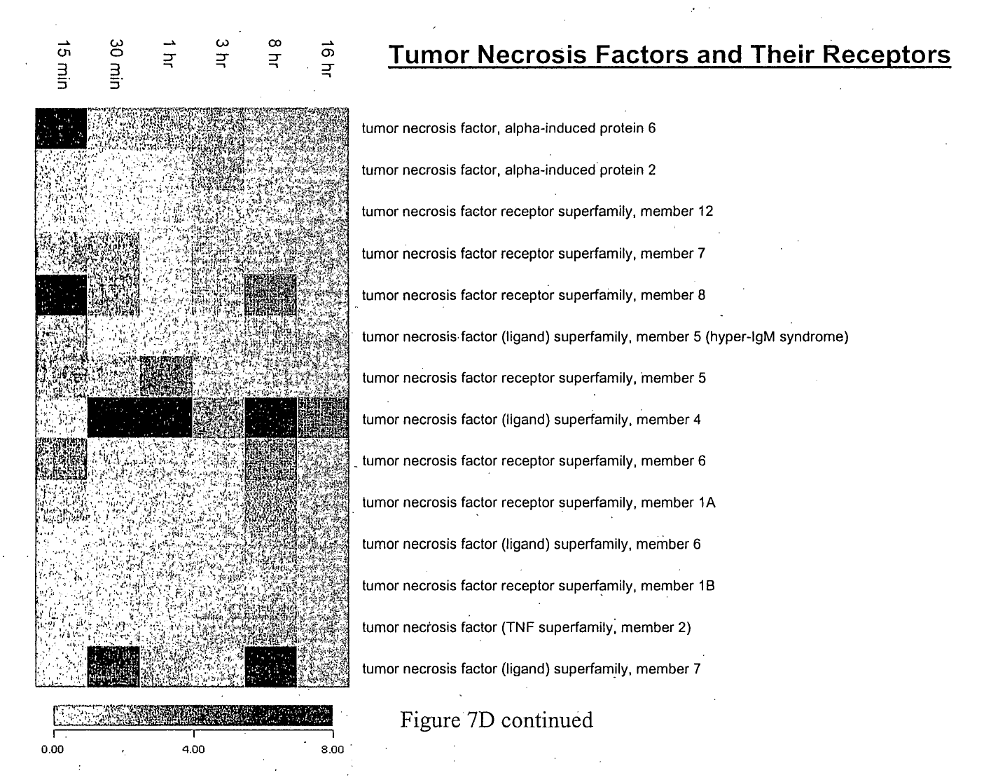

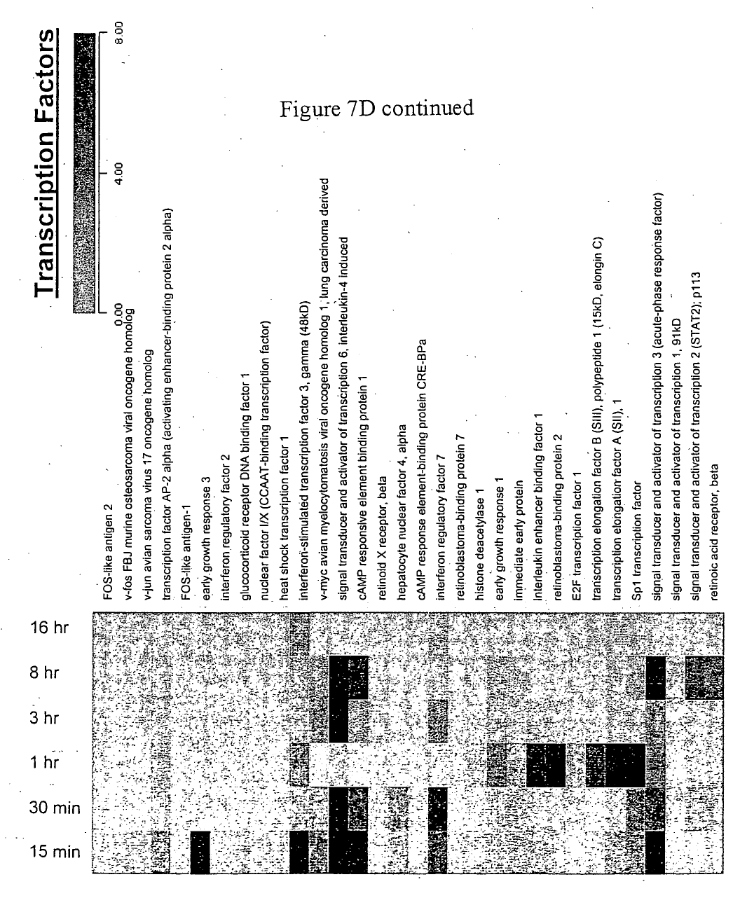

Time-dependent quantitation of the levels of gene expression in HDF cells and HEK cells is shown in FIGS. 6 and 7, respectively. The data was analyzed using a K-means test for 5 expression groups (Clusters A-E), corresponding to early to late expression. In FIGS. 6A (HDF cells) and 7A (HEK cells), line graphs show the time-dependent expression profile for each cluster of genes. Shown in FIGS. 6B-D and 7B-D are the expression profiles of the following functional families: Adhesion Molecules; Cyclins; DNA Synthesis Proteins; Growth Factors and corresponding Receptors; Interleukins; Interferons and corresponding Receptors; MAP Kinases; other Kinases; Matrix Metalloproteinases and their Inhibitors; Protein Kinase Cs; Tumor Necrosis Factors and their Receptors; and Transcription Factors. In FIGS. 6B-D (HDF cells) and 7B-D (HEK cells) genes within functional groups are listed from top to bottom based on onset of expression with early expressed genes at the top of each figure and progressively later expressed genes towards the bottom. From these findings it is clear that treatment of HDF cells with the RF field induces a large number of the genes studied in a programmed manner. The genes that showed the earliest response included genes that encode extracellular matrix proteins and signal transduction. Genes involved in regulation of the cell cycle and DNA synthesis were transcribed at later time points, corresponding to the influx of signal from the extracellular membrane through the cytoplasm, and into the nucleus.

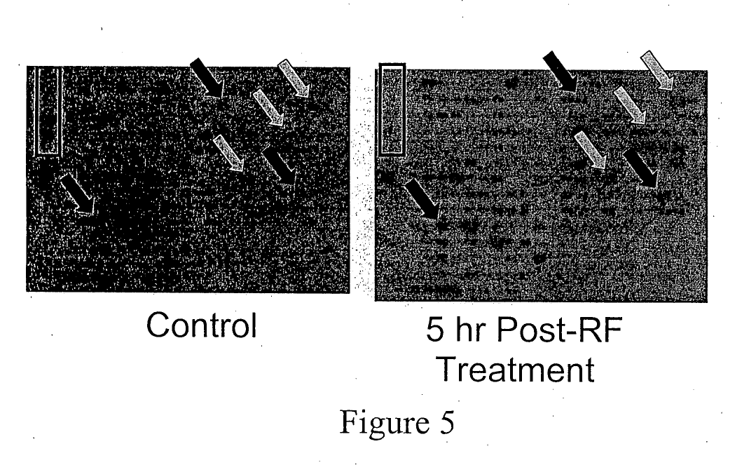

FIG. 5 shows autoradiographs from studies using the 234 gene micro-array related to inflammation response. Delivery of electromagnetic energy induced a substantial number of genes to be expressed at levels substantially greater than control. Examples of gene products that showed no response to electromagnetic energy and gene products that were induced by electromagnetic energy are indicated by the green and red arrows, respectively.

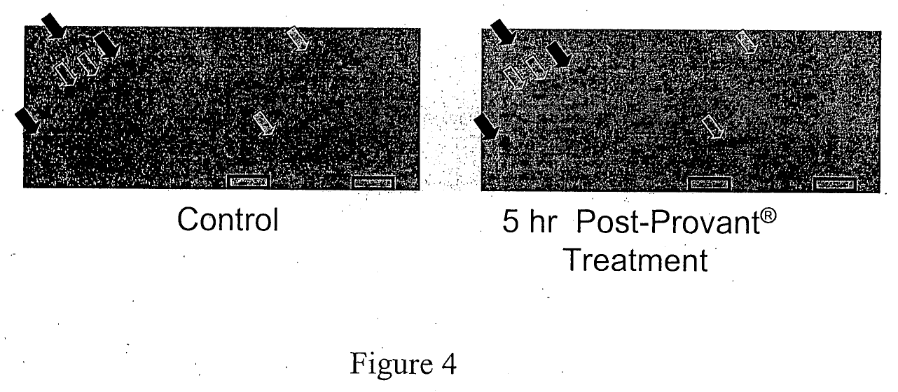

FIG. 4 shows autoradiographs from studies using the 1,176 gene micro-array related to the cell cycle and cell growth. As was found with the 234 gene micro-array, the levels of many gene products were changed after delivery of electromagnetic energy. Table 1 below sets forth the names and corresponding Genbank Accession numbers for all genes that were found to have a significant (four-fold or more) increase in expression following delivery of electromagnetic energy.

As described above, the types of gene products that are modulated by delivery of electromagnetic radiation can be classified into a number of groups including extracellular matrix receptors, signal transduction proteins, cell cycle regulators, transcription factors and DNA synthesis proteins. The results summarized in FIGS. 6 and 7 demonstrate that electromagnetic energy modulates the activity of molecular regulatory networks that mediate a number of inflammatory and cell proliferation responses such as wound healing.

TABLE 1
Genes showing at least 4-Fold Expression
Increase upon Treatment with Electromagnetic Energy

| Genbank | |
|---|---|
| Accession | |
| Number | Name |
| U18087 | 3′5′-cAMP phosphodiesterase HPDE4A6 |
| U22456 | 5′-AMP-activated protein kinase catalytic alpha-1 |
| | subunit; AMPK alpha-1 chain |
| U41766 | a disintegrin and metalloproteinase domain 9 (meltrin |
| | gamma) |
| L13738 | activated p21cdc42Hs kinase |
| U12979 | activated RNA polymerase II transcription cofactor 4 |
| U14722 | activin A receptor, type IB |
| M74088 | adenomatosis polyposis coli |
| X68486 | adenosine A2a receptor |
| X76981 | adenosine A3 receptor |
| X74210 | adenylate cyclase 2 (brain) |
| D25538 | adenylate cyclase 7 |
| AF036927 | adenylate cyclase 9 |
| M36340 | ADP-ribosylation factor 1 |
| M15169 | adrenergic, beta-2-, receptor, surface |
| AB010575 | amiloride-sensitive cation channel 3, testis |
| M20132 | androgen receptor (dihydrotestosterone receptor; |
| | testicular feminization; spinal and bulbar muscular |
| | atrophy; Kennedy disease) |
| M87290 | angiotensin receptor 1 |
| M12154 | antigen p97 (melanoma associated) identified by |
| | monoclonal antibodies 133.2 and 96.5 |
| AF013263 | apoptotic protease activating factor |
| U11700 | ATPase, Cu++ transporting, beta polypeptide (Wilson |
| | disease) |
| U51478 | ATPase, Na+/K+ transporting, beta 3 polypeptide |
| U45878 | baculoviral IAP repeat-containing 3 |
| M14745 | B-cell CLL/lymphoma 2 |
| M31732 | B-cell CLL/lymphoma 3 |
| U00115 | B-cell CLL/lymphoma 6 (zinc finger protein 51) |
| U15172 | BCL2/adenovirus E1B 19 kD-interacting protein 1 |
| U66879 | BCL2-antagonist of cell death |
| L22474 | BCL2-associated X protein |
| X89986 | BCL2-interacting killer (apoptosis-inducing) |
| Z23115 | BCL2-like 1 |
| U59747 | BCL2-like 2 |
| U29680 | BCL2-related protein A1 |
| D21878 | bone marrow stromal cell antigen 1 |
| M22491 | bone morphogenetic protein 3 (osteogenic) |
| M60315 | bone morphogenetic protein 6 |
| M97016 | bone morphogenetic protein 8 (osteogenic protein 2) |
| U76638 | BRCA1 associated RING domain 1 |
| X58957 | Bruton agammaglobulinemia tyrosine kinase |
| AF046079 | budding uninhibited by benzimidazoles 1 (yeast homolog), |
| | beta |
| Z13009 | cadherin 1, type 1, E-cadherin (epithelial) |
| X79981 | cadherin 5, type 2, VE-cadherin (vascular epithelium) |
| L00587 | calcitonin receptor |
| M94172 | calcium channel, voltage-dependent, L type, alpha 1B |
| | subunit |
| L41816 | calcium/calmodulin-dependent protein kinase I |
| L24959 | calcium/calmodulin-dependent protein kinase IV |
| M23254 | calpain 2, (m/II) large subunit |
| X04106 | calpain, small subunit 1 |
| M31630 | cAMP response element binding protein (CRE-BP1); |
| | transcription factor ATF2; HB16 |
| L05515 | cAMP response element-binding protein CRE-BPa |
| U89896 | casein kinase 1, gamma 2 |
| J02853 | casein kinase 2, alpha 1 polypeptide |
| U84388 | CASP2 and RIPK1 domain containing adaptor with death |
| | domain |
| U13699 | caspase 1, apoptosis-related cysteine protease |
| | (interleukin 1, beta, convertase) |
| U13737 | caspase 3, apoptosis-related cysteine protease |
| X87838 | catenin (cadherin-associated protein), beta 1 (88 kD) |
| M11233 | cathepsin D (lysosomal aspartyl protease) |
| X12451 | cathepsin L |
| M37197 | CCAAT-box-binding transcription factor |
| L25259 | CD86 antigen (CD28 antigen ligand 2, B7-2 antigen) |
| U05340 | CDC20 (cell division cycle 20, S. cerevisiae, homolog) |
| X54941 | CDC28 protein kinase 1 |
| X54942 | CDC28 protein kinase 2 |
| L29222 | CDC-like kinase 1 |
| U03882 | chemokine (C-C motif) receptor 2 |
| X91906 | chloride channel 5 (nephrolithiasis 2, X-linked, Dent |
| | disease) |
| M30185 | cholesteryl ester transfer protein, plasma |
| U62439 | cholinergic receptor, nicotinic, beta polypeptide 4 |
| U33286 | chromosome segregation 1 (yeast homolog)-like |
| M62424 | coagulation factor II (thrombin) receptor |
| M37435 | colony stimulating factor 1 (macrophage) |
| M11220 | colony stimulating factor 2 (granulocyte-macrophage) |
| M92934 | connective tissue growth factor |
| X56692 | C-reactive protein, pentraxin-related |
| D84657 | cryptochrome 1 (photolyase-like) |
| L13278 | crystallin, zeta (quinone reductase) |
| L12579 | cut (Drosophila)-like 1 (CCAAT displacement protein) |
| U66838 | cyclin A1 |
| AF091433 | cyclin E2 |
| D88435 | cyclin G associated kinase |
| U47413 | cyclin G1 |
| U47414 | cyclin G2 |
| U11791 | cyclin H |
| AF045161 | cyclin T1 |
| M68520 | cyclin-dependent kinase 2 |
| L25676 | cyclin-dependent kinase 9 (CDC2-related kinase) |
| U17075 | cyclin-dependent kinase inhibitor 2B (p15, inhibits CDK4) |
| L25876 | cyclin-dependent kinase inhibitor 3 (CDK2-associated dual |
| | specificity phosphatase) |
| M28668 | cystic fibrosis transmembrane conductance regulator, ATP- |
| | binding cassette (sub-family C, member 7) |
| Z00036 | cytochrome P450, subfamily I (aromatic compound- |
| | inducible), polypeptide 2 |
| J02871 | cytochrome P450, subfamily IVB, polypeptide 1 |
| M13267 | cytosolic superoxide dismutase 1 (SOD1) |
| U18321 | death associated protein 3 |
| AF015956 | death-associated protein 6 |
| X76104 | death-associated protein kinase 1 |
| M98331 | defensin, alpha 6, Paneth cell-specific |
| Z71389 | defensin, beta 2 |
| M74777 | dipeptidylpeptidase IV (CD26, adenosine deaminase |
| | complexing protein 2) |
| M60278 | diphtheria toxin receptor (heparin-binding epidermal |
| | growth factor-like growth factor) |
| X74764 | discoidin domain receptor family, member 2 |
| AF064019 | DNA fragmentation factor, 40 kD, beta polypeptide |
| | (caspase-activated DNase) |
| U91985 | DNA fragmentation factor, 45 kD, alpha polypeptide |
| X59764 | DNA-(apurinic or apyrimidinic site) lyase; AP |
| | endonuclease 1; APEX nuclease (APEN; APE1); REF-1 protein |
| D28468 | DNA-binding protein TAXREB302; albumin D box-binding |
| | protein (DBP) |
| U35835 | DNA-dependent protein kinase (DNA-PK) + DNA-PK catalytic |
| | subunit (XRCC7) |
| D49547 | DnaJ (Hsp40) homolog, subfamily B, member 1 |
| U28424 | DnaJ (Hsp40) homolog, subfamily C, member 3 |
| L11329 | dual specificity phosphatase 2 |
| Y08302 | dual specificity phosphatase 9 |
| M25269 | ELK1, member of ETS oncogene family |
| J05081 | endothelin 3 |
| L06623 | endothelin receptor type B |
| M18391 | EphA1 |
| L41939 | EphB2 |
| U14187 | ephrin-A3 |
| U14188 | ephrin-A4 |
| U12535 | epidermal growth factor receptor pathway substrate 8 |
| L05779 | epoxide hydrolase 2, cytoplasmic |
| L16464 | ets variant gene 3 |
| M31899 | excision repair cross-complementing rodent repair |
| | deficiency, complementation group 3 (xeroderma |
| | pigmentosum group B complementing) |
| L04791 | excision repair cross-complementing rodent repair |
| | deficiency, complementation group 6 |
| X60188 | extracellular signal-regulated kinase 1 (ERK1); insulin- |
| | stimulated MAP2 kinase; MAP kinase 1 (MAPK 1); p44-MAPK; |
| | microtubule-associated protein-2 kinase |
| X86779 | FAST kinase |
| X52192 | feline sarcoma (Snyder-Theilen) viral (v-fes)/Fujinami |
| | avian sarcoma (PRCII) viral (v-fps) oncogene homolog |
| M64082 | flavin containing monooxygenase 1 |
| M76673 | formyl peptide receptor-like 2 |
| X16706 | FOS-like antigen 2 |
| X16707 | FOS-like antigen-1 |
| M19922 | fructose-1,6-bisphosphatase 1 |
| X15376 | gamma-aminobutyric acid (GABA) A receptor, gamma 2 |
| L34357 | GATA-binding protein 4 |
| AF067855 | geminin |
| M95809 | general transcription factor IIH, polypeptide 1 (62 kD |
| | subunit) |
| M64752 | glutamate receptor, ionotropic, AMPA 1 |
| D28538 | glutamate receptor, metabotropic 5 |
| Y00433 | glutathione peroxidase 1 |
| X53463 | glutathione peroxidase-gastrointestinal (GSHPX-GI); |
| | glutathione peroxidase-related protein 2 (GPRP) |
| X15722 | glutathione reductase |
| X08020 | glutathione S-transferase M4 |
| L33801 | glycogen synthase kinase 3 beta |
| V00518 | glycoprotein hormones, alpha polypeptide |
| X53799 | GRO2 oncogene |
| AF078077 | growth arrest and DNA-damage-inducible, beta |
| AF078078 | growth arrest and DNA-damage-inducible, gamma |
| L29511 | growth factor receptor-bound protein 2 |
| U10550 | GTP-binding protein overexpressed in skeletal muscle |
| L22075 | guanine nucleotide binding protein (G protein), alpha 13 |
| M14631 | guanine nucleotide binding protein (G protein), alpha |
| | stimulating activity polypeptide 1 |
| AF017656 | guanine nucleotide binding protein (G protein), beta 5 |
| M36430 | guanine nucleotide binding protein (G protein), beta |
| | polypeptide 1 |
| M36429 | guanine nucleotide binding protein (G protein), beta |
| | polypeptide 2 |
| X66533 | guanylate cyclase 1, soluble, beta 3 |
| X54079 | heat shock 27 kD protein 1 |
| M11717 | heat shock 70 kD protein 1A |
| Y00371 | heat shock 70 kD protein 8 |
| M64673 | heat shock transcription factor 1 |
| X06985 | heme oxygenase (decycling) 1 |
| D21243 | heme oxygenase (decycling) 2 |
| M60718 | hepatocyte growth factor (hepapoietin A; scatter factor) |
| X76930 | hepatocyte nuclear factor 4, alpha |
| D16431 | hepatoma-derived growth factor (high-mobility group |
| | protein 1-like) |
| D14012 | HGF activator |
| M75952 | homeo box 11 (T-cell lymphoma 3-associated breakpoint) |
| U03056 | hyaluronoglucosaminidase 1 |
| AF071596 | immediate early response 3 |
| L14754 | immunoglobulin mu binding protein 2 |
| M13981 | inhibin, alpha |
| J03634 | inhibin, beta A (activin A, activin AB alpha polypeptide) |
| M97796 | inhibitor of DNA binding 2, dominant negative helix-loop- |
| | helix protein |
| X69111 | inhibitor of DNA binding 3, dominant negative helix-loop- |
| | helix protein |
| AF044195 | inhibitor of kappa light polypeptide gene enhancer in B- |
| | cells, kinase complex-associated protein |
| M10051 | insulin receptor |
| J05046 | insulin receptor-related receptor |
| M27544 | insulin-like growth factor 1 (somatomedin C) |
| X04434 | insulin-like growth factor 1 receptor |
| M29645 | insulin-like growth factor 2 (somatomedin A) |
| M31145 | insulin-like growth factor binding protein 1 |
| M31159 | insulin-like growth factor binding protein 3 |
| M14648 | integrin, alpha V (vitronectin receptor, alpha |
| | polypeptide, antigen CD51) |
| J02703 | integrin, beta 3 (platelet glycoprotein IIIa, antigen |
| | CD61) |
| J05633 | integrin, beta 5 |
| M73780 | integrin, beta 8 |
| U40282 | integrin-linked kinase |
| X14454 | interferon regulatory factor 1 |
| M28622 | interferon, beta 1, fibroblast |
| J00209 | interferon-alpha2 precursor (IFN-alpha; IFNA); leukocyte |
| | interferon-alphaA (LEIF A); roferon + IFN-alpha10 |
| | precursor; LEIF C; IFN-alpha-6L |
| K02770 | interleukin 1, beta |
| M57627 | interleukin 10 |
| U03187 | interleukin 12 receptor, beta 1 |
| M65291 | interleukin 12A (natural killer cell stimulatory factor |
| | 1, cytotoxic lymphocyte maturation factor 1, p35) |
| M74782 | interleukin 3 receptor, alpha (low affinity) |
| X04688 | interleukin 5 (colony-stimulating factor, eosinophil) |
| M75914 | interleukin 5 receptor, alpha |
| M57230 | interleukin 6 signal transducer (gp130, oncostatin M |
| | receptor) |
| Y00787 | interleukin 8 |
| M68932 | interleukin 8 receptor, alpha |
| U58198 | interleukin enhancer binding factor 1 |
| U10324 | interleukin enhancer binding factor 3, 90 kD |
| AF005216 | Janus kinase 2 (a protein tyrosine kinase) |
| U09607 | Janus kinase 3 (a protein tyrosine kinase, leukocyte) |
| AF052432 | katanin p80 (WD40-containing) subunit B 1 |
| M74387 | L1 cell adhesion molecule (hydrocephalus, stenosis of |
| | aqueduct of Sylvius 1, MASA (mental retardation, aphasia, |
| | shuffling gait and adducted thumbs) syndrome, spastic |
| | paraplegia 1) |
| X53961 | lactotransferrin |
| X61615 | leukemia inhibitory factor receptor |
| X84740 | ligase III, DNA, ATP-dependent |
| D26309 | LIM domain kinase 1 |
| AF036905 | linker for activation of T cells |
| AF055581 | lymphocyte adaptor protein |
| U07236 | lymphocyte-specific protein tyrosine kinase |
| L11015 | lymphotoxin beta (TNF superfamily, member 3) |
| S75313 | Machado-Joseph disease (spinocerebellar ataxia 3, |
| | olivopontocerebellar ataxia 3, autosomal dominant, ataxin |
| | 3) |
| X70040 | macrophage stimulating 1 receptor (c-met-related tyrosine |
| | kinase) |
| U57456 | MAD (mothers against decapentaplegic, Drosophila) homolog 1 |
| U44378 | MAD (mothers against decapentaplegic, Drosophila) homolog 4 |
| M11886 | major histocompatibility complex, class I, C |
| X57766 | matrix metalloproteinase 11 (stromelysin 3) |
| X89576 | matrix metalloproteinase 17 (membrane-inserted) |
| J03210 | matrix metalloproteinase 2 (gelatinase A, 72 kD |
| | gelatinase, 72 kD type IV collagenase) |
| D50477 | matrix metalloproteinase-16 precursor (MMP-16); membrane- |
| | type matrix metalloproteinase 3 (MT-MMP 3); MMP-X2 |
| L06895 | MAX dimerization protein |
| D21063 | MCM2 DNA replication licensing factor (nuclear protein |
| | BM28) (KIAA0030). |
| X82895 | membrane protein, palmitoylated 2 (MAGUK p55 subfamily |
| | member 2) |
| J02958 | met proto-oncogene (hepatocyte growth factor receptor) |
| D84557 | minichromosome maintenance deficient (mis5, S. pombe) 6 |
| D38073 | minichromosome maintenance deficient (S. cerevisiae) 3 |
| X74794 | minichromosome maintenance deficient (S. cerevisiae) 4 |
| X74795 | minichromosome maintenance deficient (S. cerevisiae) 5 |
| | (cell division cycle 46) |
| D55716 | minichromosome maintenance deficient (S. cerevisiae) 7 |
| L26318 | mitogen-activated protein kinase 8 |
| U25265 | mitogen-activated protein kinase kinase 5 |
| U39657 | mitogen-activated protein kinase kinase 6 |
| D14497 | mitogen-activated protein kinase kinase kinase 8 |
| U09578 | mitogen-activated protein kinase-activated protein kinase 3 |
| X72755 | monokine induced by gamma interferon |
| Z12020 | mouse double minute 2, human homolog of; p53-binding |
| | protein |
| X76538 | MpV17 transgene, murine homolog, glomerulosclerosis |
| M62397 | mutated in colorectal cancers |
| U07418 | mutL (E. coli) homolog 1 (colon cancer, nonpolyposis type |
| | 2) |
| U18840 | myelin oligodendrocyte glycoprotein |
| L08246 | myeloid cell leukemia sequence 1 (BCL2-related) |
| M81750 | myeloid cell nuclear differentiation antigen |
| M33374 | NADH-ubiquinone oxidoreductase B18 subunit; complex I-B18 |
| | (CI-B18); cell adhesion protein SQM1 |
| M81840 | neural retina leucine zipper |
| L12261 | neuregulin 1 |
| X02751 | neuroblastoma RAS viral (v-ras) oncogene homolog |
| U02081 | neuroepithelial cell transforming gene 1 |
| M60915 | neurofibromin 1 (neurofibromatosis, von Recklinghausen |
| | disease, Watson disease) |
| L11353 | neurofibromin 2 (bilateral acoustic neuroma) |
| U05012 | neurotrophic tyrosine kinase, receptor, type 3 |
| M86528 | neurotrophin 5 (neurotrophin 4/5) |
| X96586 | neutral sphingomyelinase (N-SMase) activation associated |
| | factor |
| L09210 | nitric oxide synthase 2A (inducible, hepatocytes) |
| L16785 | non-metastatic cells 2, protein (NM23B) expressed in |
| Z11583 | nuclear mitotic apparatus protein 1 |
| M24898 | nuclear receptor subfamily 1, group D, member 1 |
| X12795 | nuclear receptor subfamily 2, group F, member 1 |
| M29971 | O-6-methylguanine-DNA methyltransferase |
| M27288 | oncostatin M |
| U63717 | osteoclast stimulating factor 1 |
| U24152 | p21/Cdc42/Rac1-activated kinase 1 (yeast Ste20-related) |
| M96944 | paired box gene 5 (B-cell lineage specific activator |
| | protein) |
| L19606 | paired box gene 8 |
| D13510 | pancreatitis-associated protein |
| M31213 | papillary thyroid carcinoma-encoded protein + ret proto- |
| | oncogene |
| M63012 | paraoxonase 1 |
| M24398 | parathymosin |
| L19185 | peroxiredoxin 2 |
| U92436 | phosphatase and tensin homolog (mutated in multiple |
| | advanced cancers 1) |
| U85245 | phosphatidylinositol-4-phosphate 5-kinase, type II, beta |
| U40370 | phosphodiesterase 1A, calmodulin-dependent |
| U56976 | phosphodiesterase 1B, calmodulin-dependent |
| U02882 | phosphodiesterase 4D, cAMP-specific (dunce (Drosophila)- |
| | homolog phosphodiesterase E3) |
| Z29090 | phosphoinositide-3-kinase, catalytic, alpha polypeptide |
| U86453 | phosphoinositide-3-kinase, catalytic, delta polypeptide |
| M61906 | phosphoinositide-3-kinase, regulatory subunit, |
| | polypeptide 1 (p85 alpha) |
| X80907 | phosphoinositide-3-kinase, regulatory subunit, |
| | polypeptide 2 (p85 beta) |
| M34667 | phospholipase C, gamma 1 (formerly subtype 148) |
| X14034 | phospholipase C, gamma 2 (phosphatidylinositol-specific) |
| M54915 | pim-1 oncogene |
| X54936 | placental growth factor, vascular endothelial growth |
| | factor-related protein |
| X05199 | plasminogen |
| U08839 | plasminogen activator, urokinase receptor |
| D10202 | platelet-activating factor receptor |
| X02811 | platelet-derived growth factor beta polypeptide (simian |
| | sarcoma viral (v-sis) oncogene homolog) |
| U01038 | polo (Drosophia)-like kinase |
| L09561 | polymerase (DNA directed), epsilon |
| U24660 | potassium inwardly-rectifying channel, subfamily J, |
| | member 6 |
| Y15065 | potassium voltage-gated channel, KQT-like subfamily, |
| | member 2 |
| AF033347 | potassium voltage-gated channel, KQT-like subfamily, |
| | member 3 |
| X13403 | POU domain, class 2, transcription factor 1 |
| M36542 | POU domain, class 2, transcription factor 2 |
| X67055 | pre-alpha (globulin) inhibitor, H3 polypeptide |
| U41816 | prefoldin 4 |
| K02268 | prodynorphin |
| S85655 | prohibitin |
| D45027 | protease inhibitor 15 |
| D45248 | proteasome (prosome, macropain) activator subunit 2 (PA28 |
| | beta) |
| D88378 | proteasome (prosome, macropain) inhibitor subunit 1 |
| | (PI31) |
| D00759 | proteasome (prosome, macropain) subunit, alpha type, 1 |
| D00762 | proteasome (prosome, macropain) subunit, alpha type, 3 |
| D00763 | proteasome (prosome, macropain) subunit, alpha type, 4 |
| S76965 | protein kinase (cAMP-dependent, catalytic) inhibitor |
| | alpha |
| J03075 | protein kinase C substrate 80K-H |
| X65293 | protein kinase C, epsilon |
| M55284 | protein kinase C, eta |
| U33053 | protein kinase C-like 1 |
| M34182 | protein kinase, cAMP-dependent, catalytic, gamma |
| M33336 | protein kinase, cAMP-dependent, regulatory, type I, alpha |
| | (tissue specific extinguisher 1) |
| X14968 | protein kinase, cAMP-dependent, regulatory, type II, |
| | alpha |
| M31158 | protein kinase, cAMP-dependent, regulatory, type II, beta |
| M35663 | protein kinase, interferon-inducible double stranded RNA |
| | dependent |
| M63960 | protein phosphatase 1, catalytic subunit, alpha isoform |
| S87759 | protein phosphatase 1A (formerly 2C), magnesium- |
| | dependent, alpha isoform |
| X12646 | protein phosphatase 2 (formerly 2A), catalytic subunit, |
| | alpha isoform |
| M64929 | protein phosphatase 2 (formerly 2A), regulatory subunit B |
| | (PR 52), alpha isoform |
| M64930 | protein phosphatase 2 (formerly 2A), regulatory subunit B |
| | (PR 52), beta isoform |
| L14778 | protein phosphatase 3 (formerly 2B), catalytic subunit, |
| | alpha isoform (calcineurin A alpha) |
| M30773 | protein phosphatase 3 (formerly 2B), regulatory subunit B |
| | (19 kD), alpha isoform (calcineurin B, type I) |
| U48296 | protein tyrosine phosphatase type IVA, member 1 |
| L08807 | protein tyrosine phosphatase, non-receptor type 11 |
| L09247 | protein tyrosine phosphatase, receptor type, G |
| M93426 | protein tyrosine phosphatase, receptor-type, Z |
| | polypeptide 1 |
| M35203 | protein-tyrosine kinase (JAK1) |
| L34583 | protein-tyrosine phosphatase 1E |
| M26708 | prothymosin, alpha (gene sequence 28) |
| L13616 | PTK2 protein tyrosine kinase 2 |
| U33635 | PTK7 protein tyrosine kinase 7 |
| Y07701 | PUROMYCIN-SENSITIVE AMINOPEPTIDASE (EC 3.4.11.-) (PSA) |
| D10923 | putative chemokine receptor; GTP-binding protein |
| L42450 | pyruvate dehydrogenase kinase, isoenzyme 1 |
| L42379 | quiescin Q6 |
| M28210 | RAB3A, member RAS oncogene family |
| M28211 | RAB4, member RAS oncogene family |
| M28215 | RAB5A, member RAS oncogene family |
| M28212 | RAB6A, member RAS oncogene family |
| U63139 | RAD50 (S. cerevisiae) homolog |
| X82260 | Ran GTPase activating protein 1 |
| M64788 | RAP1, GTPase activating protein 1 |
| X63465 | RAP1, GTP-GDP dissociation stimulator 1 |
| M22995 | RAP1A, member of RAS oncogene family |
| X08004 | RAP1B, member of RAS oncogene family |
| X06820 | ras homolog gene family, member B |
| M23379 | RAS p21 protein activator (GTPase activating protein) 1 |
| L26584 | Ras protein-specific guanine nucleotide-releasing factor 1 |
| L24564 | Ras-related associated with diabetes |
| M29870 | ras-related C3 botulinum toxin substrate 1 (rho family, |
| | small GTP binding protein Rac1) |
| X93499 | ras-related protein RAB-7 |
| M97675 | receptor tyrosine kinase-like orphan receptor 1 |
| D10232 | renin-binding protein |
| L07541 | replication factor C (activator 1) 3 (38 kD) |
| M87339 | replication factor C (activator 1) 4 (37 kD) |
| L07493 | replication protein A3 (14 kD) |
| M15400 | retinoblastoma 1 (including osteosarcoma) |
| S66431 | retinoblastoma-binding protein 2 |
| X74594 | retinoblastoma-like 2 (p130) |
| X07282 | retinoic acid receptor, beta |
| M84820 | retinoid X receptor, beta |
| U17032 | Rho GTPase activating protein 5 |
| U02082 | Rho guanine nucleotide exchange factor (GEF) 5 |
| X56932 | ribosomal protein L13a |
| X69391 | ribosomal protein L6 |
| L07597 | ribosomal protein S6 kinase, 90 kD, polypeptide 1 |
| U23946 | RNA binding motif protein 5 |
| X58079 | S100 calcium-binding protein A1 |
| M86757 | S100 calcium-binding protein A7 (psoriasin 1) |
| X06234 | S100 calcium-binding protein A8 (calgranulin A) |
| U01160 | sarcoma amplified sequence |
| Y00757 | secretory granule, neuroendocrine protein 1 (7B2 protein) |
| U60800 | sema domain, immunoglobulin domain (Ig), transmembrane |
| | domain (TM) and short cytoplasmic domain, (semaphorin) 4D |
| M14091 | serine (or cysteine) proteinase inhibitor, clade A |
| | (alpha-1 antiproteinase, antitrypsin), member 7 |
| U04313 | serine (or cysteine) proteinase inhibitor, clade B |
| | (ovalbumin), member 5 |
| L40377 | serine (or cysteine) proteinase inhibitor, clade B |
| | (ovalbumin), member 8 |
| U71364 | serine (or cysteine) proteinase inhibitor, clade B |
| | (ovalbumin), member 9 |
| X04429 | serine (or cysteine) proteinase inhibitor, clade E |
| | (nexin, plasminogen activator inhibitor type 1), member 1 |
| Z81326 | serine (or cysteine) proteinase inhibitor, clade I |
| | (neuroserpin), member 1 |
| U78095 | serine protease inhibitor, Kunitz type, 2 |
| AF008552 | serine/threonine kinase 12 |
| L20321 | serine/threonine kinase 2 |
| D84212 | serine/threonine kinase 6 |
| M97935 | signal transducer and activator of transcription 1, 91 kD |
| M97934 | signal transducer and activator of transcription 2 |
| | (STAT2); p113 |
| L29277 | signal transducer and activator of transcription 3 |
| | (acute-phase response factor) |
| M57502 | small inducible cytokine A1 (I-309, homologous to mouse |
| | Tca-3) |
| J04130 | small inducible cytokine A4 (homologous to mouse Mip-1b) |
| M21121 | small inducible cytokine A5 (RANTES) |
| AJ002211 | small inducible cytokine B subfamily (Cys-X-Cys motif), |
| | member 13 (B-cell chemoattractant) |
| U46767 | small inducible cytokine subfamily A (Cys-Cys), member 13 |
| X02530 | small inducible cytokine subfamily B (Cys-X-Cys), member |
| | 10 |
| X78686 | small inducible cytokine subfamily B (Cys-X-Cys), member |
| | 5 (epithelial-derived neutrophil-activating peptide 78) |
| U10117 | small inducible cytokine subfamily E, member 1 |
| | (endothelial monocyte-activating) |
| M21940 | S-mephenytoin 4 hydroxylase; cytochrome P450 IIC9 |
| | (CYP2C9) + CYP2C10 + CYP2C17 + CYP2C18 + CYP2C19 |
| AF068920 | soc-2 (suppressor of clear, C. elegans) homolog |
| M77235 | sodium channel, voltage-gated, type V, alpha polypeptide |
| | (long (electrocardiographic) QT syndrome 3) |
| D00099 | SODIUM/POTASSIUM-TRANSPORTING ATPASE ALPHA-1 CHAIN (EC |
| | 3.6.1.37) (SODIUM PUMP) (NA+/K+ ATPASE). |
| L14595 | solute carrier family 1 (glutamate/neutral amino acid |
| | transporter), member 4 |
| U03506 | solute carrier family 1 (neuronal/epithelial high |
| | affinity glutamate transporter, system Xag), member 1 |
| U13173 | solute carrier family 15 (oligopeptide transporter), |
| | member 1 |
| L31801 | solute carrier family 16 (monocarboxylic acid |
| | transporters), member 1 |
| U10554 | solute carrier family 18 (vesicular acetylcholine), |
| | member 3 |
| L09118 | solute carrier family 18 (vesicular monoamine), member 2 |
| U14528 | solute carrier family 26 (sulfate transporter), member 2 |
| AF025409 | solute carrier family 30 (zinc transporter), member 4 |
| M95549 | solute carrier family 5 (sodium/glucose cotransporter), |
| | member 2 |
| M95167 | solute carrier family 6 (neurotransmitter transporter, |
| | dopamine), member 3 |
| S75989 | solute carrier family 6 (neurotransmitter transporter, |
| | GABA), member 11 |
| S70609 | solute carrier family 6 (neurotransmitter transporter, |
| | glycine), member 9 |
| S80071 | solute carrier family 6 (neurotransmitter transporter, L- |
| | proline), member 7 |
| AJ000730 | solute carrier family 7 (cationic amino acid transporter, |
| | y+ system), member 4 |
| AF077866 | solute carrier family 7 (cationic amino acid transporter, |
| | y+ system), member 5 |
| M81768 | solute carrier family 9 (sodium/hydrogen exchanger), |
| | isoform 1 (antiporter, Na+/H+, amiloride sensitive) |
| L13857 | son of sevenless (Drosophila) homolog 1 |
| M97190 | Sp2 transcription factor |
| AF039843 | sprouty (Drosophila) homolog 2 |
| U08098 | sulfotransferase, estrogen-preferring |
| AF069734 | suppressor of Ty (S. cerevisiae) 3 homolog |
| AF046873 | synapsin III |
| X07024 | TATA box binding protein (TBP)-associated factor, RNA |
| | polymerase II, A, 250 kD |
| D29767 | tec protein tyrosine kinase |
| M16552 | thrombomodulin |
| M92381 | thymosin, beta 10 |
| M17733 | thymosin, beta 4, X chromosome |
| U76456 | tissue inhibitor of metalloproteinase 4 |
| X69490 | titin |
| J03250 | topoisomerase (DNA) I |
| U59863 | TRAF family member-associated NFKB activator |
| M80627 | transcription factor 12 (HTF4, helix-loop-helix |
| | transcription factors 4) |
| M36711 | transcription factor AP-2 alpha (activating enhancer- |
| | binding protein 2 alpha) |
| L23959 | transcription factor Dp-1 |
| U18422 | transcription factor Dp-2 (E2F dimerization partner 2) |
| AF009353 | transcriptional intermediary factor 1 |
| J03241 | transforming growth factor, beta 3 |
| L07594 | transforming growth factor, beta receptor III |
| | (betaglycan, 300 kD) |
| X95384 | translational inhibitor protein p14.5 |
| U78773 | tripartite motif-containing 28 |
| U04811 | trophinin |
| X52836 | tryptophan hydroxylase (tryptophan 5-monooxygenase) |
| X75621 | tuberous sclerosis 2 |
| U57059 | tumor necrosis factor (ligand) superfamily, member 10 |
| D38122 | tumor necrosis factor (ligand) superfamily, member 6 |
| X01394 | tumor necrosis factor (TNF superfamily, member 2) |
| M32315 | tumor necrosis factor receptor superfamily, member 1B |
| M14694 | tumor protein p53 (Li-Fraumeni syndrome) |
| U82130 | tumor susceptibility gene 101 |
| D17517 | TYRO3 protein tyrosine kinase |
| X57346 | tyrosine 3-monooxygenase/tryptophan 5-monooxygenase |
| | activation protein, beta polypeptide |
| L20422 | tyrosine 3-monooxygenase/tryptophan 5-monooxygenase |
| | activation protein, eta polypeptide |
| M26880 | ubiquitin C |
| M32977 | vascular endothelial growth factor |
| U43142 | vascular endothelial growth factor C |
| V00574 | v-Ha-ras Harvey rat sarcoma viral oncogene homolog |
| AF055377 | v-maf musculoaponeurotic fibrosarcoma (avian) oncogene |
| | homolog |
| J00119 | v-mos Moloney murine sarcoma viral oncogene homolog |
| M15024 | v-myb avian myeloblastosis viral oncogene homolog |
| X66087 | v-myb avian myeloblastosis viral oncogene homolog-like 1 |
| X13293 | v-myb avian myeloblastosis viral oncogene homolog-like 2 |
| V00568 | v-myc avian myelocytomatosis viral oncogene homolog |
| L15409 | von Hippel-Lindau syndrome |
| X03484 | v-raf-1 murine leukemia viral oncogene homolog 1 |
| X15014 | v-ral simian leukemia viral oncogene homolog A (ras |
| | related) |
| M35416 | v-ral simian leukemia viral oncogene homolog B (ras |
| | related; GTP binding protein) |
| X75042 | v-rel avian reticuloendotheliosis viral oncogene homolog |
| L19067 | v-rel avian reticuloendotheliosis viral oncogene homolog |
| | A (nuclear factor of kappa light polypeptide gene |
| | enhancer in B-cells 3 (p65)) |
| M34353 | v-ros avian UR2 sarcoma virus oncogene homolog 1 |
| M16038 | v-yes-1 Yamaguchi sarcoma viral related oncogene homolog |
| U10564 | weel+ (S. pombe) homolog |
| X51630 | Wilms tumor 1 |
| Z71621 | wingless-type MMTV integration site family, member 2B |
| D21089 | xeroderma pigmentosum, complementation group C |
| M36089 | X-ray repair complementing defective repair in Chinese |
| | hamster cells 1 |
| M30938 | X-ray repair complementing defective repair in Chinese |
| | hamster cells 5 (double-strand-break rejoining; Ku |
| | autoantigen, 80 kD) |
| M76541 | YY1 transcription factor |
| D26121 | ZFM1 protein alternatively spliced product |
| M28372 | zinc finger protein 9 (a cellular retroviral nucleic acid |
| | binding protein) |
| X59738 | zinc finger X-chromosomal protein (ZFX) |
| X94991 | zyxin |

Throughout this application various patent and non-patent publications have been referenced. The disclosures of these publications in their entireties are hereby incorporated by reference in this application in order to more fully describe the state of the art to which this invention pertains.

The term “comprising” is intended herein to be open-ended, including not only the recited elements, but further encompassing any additional elements.

Although the invention has been described with reference to the examples provided above, it should be understood that various modifications can be made without departing from the spirit of the invention. Accordingly, the invention is limited only by the claims.

1. A method for accelerating the cell cycle, comprising delivering to a cell an effective amount of electromagnetic energy to accelerate the cell cycle of said cell.

2. The method of claim 1, wherein the rate at which said cell replicates its DNA increases.

3. The method of claim 1, wherein the G₁ stage of said cell cycle is shortened.

4. The method of claim 1, wherein said cell cycle is accelerated 2 fold.

5. The method of claim 1, wherein said electromagnetic energy has a wavelength in a region of the spectrum selected from the group consisting of X-ray radiation, ultraviolet radiation, visible radiation, infrared radiation, microwave radiation and radiofrequency radiation.

6. The method of claim 1, wherein said electromagnetic energy comprises an energy that is in the range of 1 to 300 mW/cm².

7. The method of claim 1, wherein said electromagnetic energy is pulsed.

8. The method of claim 1, wherein said cell is selected from the group consisting of a comprises a cell selected form the group consisting of fibroblast, neuronal cell, epitheleal cell, macrophage, neutrophil, keratinocyte, endothelial cell, epidermal melanocyte, hair follicle papilla cell, skeletal muscle cell, smooth muscle cell, osteoblast, neuron, chondrocyte, hepatocyte, pancreatic cell, kidney cell, aortic cell, bronchial cell and tracheal cell.

9. The method of claim 1, further comprising delivering to said cell an effective amount of electromagnetic energy to activate a cell cycle regulator.

10. The method of claim 1, further comprising delivering to said cell an effective amount of electromagnetic energy to activate a signal transduction protein.

11. The method of claim 1, further comprising delivering to said cell an effective amount of electromagnetic energy to activate a transcription factor.

12. The method of claim 1, further comprising delivering to said cell an effective amount of electromagnetic energy to activate a DNA synthesis protein.

13. The method of claim 1, further comprising delivering to said cell an effective amount of electromagnetic energy to activate a receptor.

14. The method of claim 1, further comprising delivering to said cell an effective amount of electromagnetic energy to inhibit the Angiotensin Receptor.

15. A method for activating a cell cycle regulator, comprising delivering to a cell an effective amount of electromagnetic energy to activate said cell cycle regulator.

16. The method of claim 15, wherein said cell cycle regulator accelerates the cell cycle of said cell.

17. The method of claim 16, wherein the rate at which said cell replicates its DNA increases.

18. The method of claim 16, wherein the G₁ stage of said cell cycle is shortened.

19. The method of claim 16, wherein said cell cycle is accelerated 2 fold.

20. The method of claim 15, wherein said electromagnetic energy has a wavelength in a region of the spectrum selected from the group consisting of X-ray radiation, ultraviolet radiation, visible radiation, infrared radiation, microwave radiation and radiofrequency radiation.

21. The method of claim 15, wherein said electromagnetic energy comprises an energy that is in the range of 1 to 300 mW/cm².

22. The method of claim 15, wherein said electromagnetic energy is pulsed.

23. The method of claim 15, wherein said cell is selected from the group consisting of a fibroblast, neuronal cell, epitheleal cell, macrophage, neutrophil, keratinocyte, endothelial cell, epidermal melanocyte, hair follicle papilla cell, skeletal muscle cell, smooth muscle cell, osteoblast, neuron, chondrocyte, hepatocyte, pancreatic cell, kidney cell, aortic cell, bronchial cell and tracheal cell.

24. A method for activating a signal transduction protein, comprising delivering to a cell an effective amount of electromagnetic energy to activate said signal transduction protein.

25. A method for activating a transcription factor, comprising delivering to a cell an effective amount of electromagnetic energy to activate said transcription factor.

26. A method for activating a DNA synthesis protein, comprising delivering to a cell an effective amount of electromagnetic energy to activate said DNA synthesis protein.

27. A method for activating a receptor, comprising delivering to a cell an effective amount of electromagnetic energy to activate said receptor.

28. A method for inhibiting an angiotensin receptor, comprising delivering to a cell an effective amount of electromagnetic energy to inhibit said angiotensin receptor.

29. A method for reducing inflammation, comprising delivering to a tissue undergoing inflammation an effective amount of electromagnetic energy to reduce said inflammation.

30. The method of claim 29, wherein said tissue undergoing inflammation comprises neuronal tissue.

31. The method of claim 30, wherein said inflammation is associated with a neuroinflammatory disease.

32. The method of claim 31, wherein said neuroinflammatory disease is a demyelinating neuroinflammatory disease.

33. A method for replacing damaged neuronal tissue, comprising delivering to a damaged neuronal tissue an effective amount of electromagnetic energy to stimulate replacement of damaged neurons.

34. A method for stimulating growth of administered cells, comprising the steps of:
(a) administering a population of cells to an individual, and
(b) delivering to said population an effective amount of electromagnetic energy to stimulate growth of said population.

35. The method of claim 34, wherein said population forms a tissue.

36. The method of claim 34, wherein said population of cells comprises a cell selected form the group consisting of fibroblast, neuronal cell, epitheleal cell, macrophage, neutrophil, keratinocyte, endothelial cell, epidermal melanocyte, hair follicle papilla cell, skeletal muscle cell, smooth muscle cell, osteoblast, neuron, chondrocyte, hepatocyte, pancreatic cell, kidney cell, aortic cell, bronchial cell and tracheal cell.

37. The method of claim 34, wherein said population of cells is administered to a wound.

38. The method of claim 34, wherein said population of cells comprises neurons.

39. The method of claim 37, wherein said population of cells is administered to a site of neuronal damage.

40. A method for stimulating formation of a tissue, comprising the steps of:
(a) contacting a population of cells with a matrix under conditions suitable for tissue formation by said cells, and
(b) delivering to said population an effective amount of electromagnetic energy to stimulate formation of said tissue.

41. The method of claim 40, wherein said matrix comprises a synthetic material.

42. The method of claim 40, wherein said matrix comprises a biological material.

43. The method of claim 40, wherein steps (a) and (b) occur ex vivo.

44. The method of claim 40, wherein said tissue comprises artificial skin.

45. The method of claim 40, further comprising a step of administering said tissue to an individual.

46. The method of claim 40, wherein said tissue is administered to a wound.

47. The method of claim 40, wherein step (b) occurs in vivo.

48. The method of claim 40, wherein steps (a) and (b) occur in vivo.

49. The method of claim 40, wherein said population of cells comprises fibroblasts or epithelial cells.

50. The method of claim 40, wherein said population of cells is administered to a wound.

51. The method of claim 40, wherein said population of cells comprises neurons.

52. The method of claim 40, wherein said population of cells is administered to a site of neuronal damage.

53. A method for monitoring progress of electromagnetic therapy, comprising detecting a level of a cell cycle regulator in a cell population following delivery to said cell population of electromagnetic energy, whereby the level of said cell cycle regulator correlates with the effectiveness of said therapy.

54. A method for modifying electromagnetic therapy, comprising monitoring progress of electromagnetic therapy according to claim 53 and modifying said electromagnetic therapy based on said level of said cell cycle regulator in said cell population.

55. A method for monitoring progress of electromagnetic therapy, comprising detecting a level of a signal transduction protein in a cell population following delivery to said cell population of electromagnetic energy, whereby the level of said signal transduction protein correlates with the effectiveness of said therapy.

56. A method for modifying electromagnetic therapy, comprising monitoring progress of electromagnetic therapy according to claim 55 and modifying said electromagnetic therapy based on said level of said signal transduction protein in said cell population.

57. A method for monitoring progress of electromagnetic therapy, comprising detecting a level of a transcription factor in a cell population following delivery to said cell population of electromagnetic energy, whereby the level of said transcription factor correlates with the effectiveness of said therapy.

58. A method for modifying electromagnetic therapy, comprising monitoring progress of electromagnetic therapy according to claim 57 and modifying said electromagnetic therapy based on said level of said transcription factor in said cell population.

59. A method for monitoring progress of electromagnetic therapy, comprising detecting a level of a DNA synthesis protein in a cell population following delivery to said cell population of electromagnetic energy, whereby the level of said DNA synthesis protein correlates with the effectiveness of said therapy.

60. A method for modifying electromagnetic therapy, comprising monitoring progress of electromagnetic therapy according to claim 59 and modifying said electromagnetic therapy based on said level of said DNA synthesis protein in said cell population.

61. A method for monitoring progress of electomagnetic therapy, comprising detecting a level of a receptor in a cell population following delivery to said cell population of electromagnetic energy, whereby the level of said receptor correlates with the effectiveness of said therapy.

62. A method for modifying electromagnetic therapy, comprising monitoring progress of electromagnetic therapy according to claim 61 and modifying said electromagnetic therapy based on said level of said receptor in said cell population.
